SEQUENCE LISTING (10 sequences)

SEQ ID NO: 1 - protein, 10 aa, synthetic construct
TPGPGVRYPL

SEQ ID NO: 2 - protein, 9 aa, synthetic construct
ALAAAAAAV

SEQ ID NO: 3 - protein, 9 aa, synthetic construct
ATAAAAAAK

SEQ ID NO: 4 - protein, 9 aa, synthetic construct
AYAAAAAAL

SEQ ID NO: 5 - protein, 9 aa, synthetic construct
APAAAAAAV

SEQ ID NO: 6 - protein, 9 aa, synthetic construct
RYAAAAALL

SEQ ID NO: 7 - protein, 8 aa, synthetic construct
ASYAAAAV

SEQ ID NO: 8 - protein, 8 aa, synthetic construct
TSYKFESV

SEQ ID NO: 9 - DNA, 11 bp, synthetic construct
  source 1..11: mol_type = other DNA
aaaaaaaaaaa

SEQ ID NO: 10 - DNA, 13 bp, synthetic construct
  source 1..13: mol_type = other DNA
ttttttttttttt

(19) United States
(12) Patent Application Publication
(10) Pub. No.: US 2025/0147038 A1
(43) Pub. Date: May 8, 2025

(54) COMPOSITIONS AND METHODS FOR CHARACTERIZING BINDING CHARACTERISTICS OF ANTIGEN BINDING MOLECULES FROM SINGLE CELLS

(71) Applicant: 10x Genomics, Inc., Pleasanton, CA (US)

(72) Inventors: Michael John Terry Stubbington, Cambridge (GB); Ravi Ramenani, Pleasanton, CA (US); Wyatt James McDonnell, Pleasanton, CA (US)

(21) Appl. No.: 19/015,083

(22) Filed: Jan. 9, 2025

Related U.S. Application Data
(63) Continuation of application No. PCT/US2023/070049, filed on Jul. 12, 2023.
(60) Provisional application No. 63/388,847, filed on Jul. 13, 2022.

(51) Int. Cl. G01N 33/68; C12N 15/10

(52) U.S. Cl. CPC G01N 33/6845; C12N 15/1065

(57) ABSTRACT

The present disclosure relates generally to compositions, methods, and systems for the characterization of binding characteristics of antigen binding molecules, e.g., antibodies, using single-cell immune profiling methodologies. The compositions, methods and systems described herein permit rapid, high-throughput identification and characterization of antigen binding molecules having desired properties.

### CROSS-REFERENCE TO RELATED APPLICATIONS

This application is a continuation of International Application No. PCT/US2023/070049, filed Jul. 12, 2023, which claims the benefit of and priority to U.S. Provisional Patent Application No. 63/388,847, filed Jul. 13, 2022, the entire contents of which are incorporated herein by reference for all purposes.

### REFERENCE TO AN ELECTRONIC SEQUENCE LISTING

The contents of the electronic sequence listing (900299.436C1_SEQUENCE_LISTING.xml; Size: 9,784 bytes; and Date of Creation: Oct. 3, 2023) is herein incorporated by reference in its entirety.

### BACKGROUND

High-throughput methods of retrieving immune receptors and their associated antigen specificity. e.g., by sequencing, provide opportunities for rapid identification of ABMs. Identification of ABMs from, e.g., human samples using such high-throughput methods is advantageous as the ABMs are not only rapidly identifiable by the methods, but, if the identified ABMs are for potential use as a biotherapeutic, are less likely to provoke an immune response in a subject (having been derived from a product of natural human immune selection and maturation). However, the interpretation of the sequencing readout to infer binding characteristics of the identified ABMs for their target antigen remain a challenge.

There is a need for improvements to methods of characterizing ABMs that specifically bind target antigens of interest with binding characteristics that are able to be accurately determined.

### SUMMARY

Provided herein are, inter alia, methods, systems and kits useful for characterization of binding characteristics of antigen-binding molecules (ABMs), such as B cell receptors (BCRs) antibodies (Abs) and antigen-binding fragments of antibodies. Understanding the binding characteristics of ABMs having desirable properties, e.g., that recognize and bind to cells displaying tumor or viral antigens, can be useful in the development of new immunotherapies to treat cancers and/or infectious disease. Also provided in some embodiments of the disclosure are kits for the discovery and/or characterization of these binding characteristics of ABMs, e.g., BCRs, antibodies or antigen-binding fragments of antibodies.

In an aspect, the present disclosure provides a method comprising: (a) partitioning a reaction mixture, or a portion thereof, into a plurality of partitions, wherein the reaction mixture comprises: (i) a plurality of pre-determined antigen binding molecule (PABM)-associated beads, wherein a first PABM-associated bead of the plurality of PABM-associated beads comprises a first PABM, and a first PABM reporter oligonucleotide comprising a first PABM barcode sequence identifying the first PABM, and (ii) a plurality of antigens comprising a first antigen operatively coupled to a first antigen reporter oligonucleotide comprising a first antigen reporter barcode sequence, wherein the first antigen binds to the first PABM of the PABM-associated bead, and wherein the partitioning provides (i) a first partition comprising the first PABM-associated bead, (ii) the first antigen, and (iii) a plurality of first partition nucleic acid barcode molecules comprising a first partition-specific barcode sequence; and generating (i) a first barcoded polynucleotide comprising (i) the first partition barcode sequence or a reverse complement thereof and (ii) the first antigen reporter barcode sequence or reverse complement thereof; and (ii) a second barcoded polynucleotide comprising (i) the first partition barcode sequence or reverse complement thereof and (ii) the first PABM reporter barcode sequence or reverse complement thereof.

In one embodiment, the reaction mixture further comprises a plurality of immune cells. In one embodiment, the plurality of immune cells comprises B cells. In one embodiment, the plurality of immune cells comprises T cells. In one embodiment, the plurality of immune cells comprises T cells and B cells.

In one embodiment, the partitioning further provides a second partition comprising (i) an immune cell expressing an antigen binding molecule (ABM), (ii) a target antigen operatively coupled to a target antigen reporter oligonucleotide comprising a target antigen barcode sequence, and (iii) a plurality of second partition nucleic acid barcode molecules comprising a second partition-specific barcode sequence. In one embodiment, the ABM is a T cell receptor (TCR). In one embodiment, the ABM is a B cell receptor (BCR), an antibody (Ab) or antigen-binding fragment thereof.

In one embodiment, the first antigen, the target antigen, or both the first antigen and the target antigen are of a pathogen, tumor, or an autoantigen. In one embodiment, the target antigen is of the pathogen, wherein the pathogen is a virus, bacteria or parasite. In one embodiment, the first antigen is of the tumor, and wherein the first antigen of the tumor is of a growth factor or a growth factor receptor. In one embodiment, the target antigen is different from the first antigen. In one embodiment, the target antigen is the same as the first antigen.

In one embodiment, the first antigen comprises a first MHC molecule bound to a first antigenic peptide.

In some embodiments, (b) further comprises generating (iii) a third barcoded polynucleotide comprising (i) the second partition barcode sequence or a reverse complement thereof and (ii) the target antigen reporter barcode sequence or a reverse complement thereof, and (iv) a fourth barcoded polynucleotide comprising (i) the second partition barcode sequence or a reverse complement thereof and (ii) a first nucleic acid sequence encoding at least a portion of the ABM expressed by the immune cell, or a reverse complement thereof.

In one embodiment, the plurality of PABM associated beads comprises a second PABM-associated bead, wherein the second PABM-associated bead comprises a second PABM and a second PABM reporter oligonucleotide comprising a second PABM barcode sequence identifying the second PABM, and wherein the partitioning provides (i) a 3^{rd} partition comprising the second PABM-associated bead, and (ii) a plurality of third partition nucleic acid barcode molecules comprising a third partition-specific barcode sequence, and wherein (b) further comprises generating a 5th barcoded polynucleotide comprising the 3^{rd} partition barcode sequence or reverse complement thereof and the 2^{nd} PABM reporter barcode sequence or reverse complement thereof. In one embodiment, the second PABM binds the first antigen, the third partition further comprises the first antigen operatively coupled to the first antigen reporter oligonucleotide, and wherein (b) further comprises generating a 6th barcoded polynucleotide comprising the 3^{rd} partition barcode sequence or reverse complement thereof and the first antigen reporter barcode sequence or reverse complement thereof. In one embodiment, the second PABM binds a second antigen, the third partition further comprises the second antigen operatively coupled to a second antigen reporter oligonucleotide comprising a second antigen reporter barcode sequence, and wherein (b) further comprises generating a 6th barcoded polynucleotide comprising the 3^{rd} partition barcode sequence or reverse complement thereof and the second antigen reporter barcode sequence or reverse complement thereof.

In one embodiment, the first PABM binds to the first antigen with a known binding characteristic. In one embodiment, the method further comprises generating a count matrix, wherein the count matrix includes an association of partition barcode sequences with first PABM barcode sequences and a quantity of first antigen barcode sequences. In one embodiment, the method further comprises using the count matrix to correlate the known binding characteristic of the first PABM for the first antigen with the quantity of first antigen barcode sequences.

In one embodiment, the second PABM binds to the first antigen or the second antigen with a known binding characteristic. In one embodiment, the known binding characteristic of the second PABM for the first antigen or the second antigen is different from that of the first PABM for the first antigen. In one embodiment, (i) the count matrix further includes an association of partition barcode sequences with second PABM barcode sequences and quantity of first or second antigen barcode sequences or (ii) the method further comprises generating a count matrix that includes an association of partition barcode sequences with second PABM barcode sequences and quantity of first or second antigen barcode sequences.

In one embodiment, the method further comprises using the count matrix to correlate the known binding characteristic of the second PABM for the first antigen or the second antigen with the quantity of first or second antigen barcode sequences.

In one embodiment, the correlations between the known binding characteristics of the 1^{st} and 2^{nd} PABMs with their associated quantities of 1^{st} or 2^{nd} antigen barcode sequences is used to infer a relationship between antigen barcode sequence quantity and the binding characteristic.]

In one embodiment, (i) the count matrix further includes an association of partition barcode sequences with target antigen UMI counts and nucleic acid sequences encoding the ABM expressed by the immune cell, or (ii) the method further comprises generating a count matrix that includes an association of partition barcode sequences with target antigen UMI counts and nucleic acid sequences encoding the ABM expressed by the immune cell.

In one embodiment, the method further comprises using the inferred relationship to estimate the binding characteristic of the ABM for the target antigen.

In some embodiments, a first nucleic acid barcode molecule of the plurality of second partition nucleic acid barcode molecules further comprises a capture sequence configured to couple to: (a) an mRNA or DNA analyte or (b) non-templated nucleotides appended to a cDNA reverse transcribed from an mRNA analyte. In one embodiment, the capture sequence is configured to couple to the mRNA analyte. In one embodiment, the capture sequence configured to couple to the mRNA analyte comprises a polyT, a gene-specific or a random sequence. In one embodiment, the capture sequence of the first nucleic acid barcode molecule is configured to couple to (b) the non-templated nucleotides appended to the cDNA reverse transcribed from the mRNA analyte. In one embodiment, the mRNA analyte is reverse transcribed to the cDNA utilizing a primer comprising a polyT sequence, a gene-specific or a random sequence. In one embodiment, the non-templated nucleotides appended to the cDNA comprise a cytosine. In one embodiment, the capture sequence configured to couple to the cDNA comprises a guanine. In one embodiment, coupling of the capture sequence to the non-templated cytosine extends reverse transcription of the cDNA into the first nucleic acid barcode molecule to generate the first barcoded nucleic molecule. In one embodiment, the first nucleic acid barcode molecule further comprises a template switch oligonucleotide (TSO).

In some embodiments, the method further comprises determining the nucleic acid sequence encoding at least the portion of the ABM from the fourth barcoded polynucleotide. In one embodiment, the nucleic acid sequence encoding at least the portion of the ABM comprises one or more of a complementarity determining region (CDR), a framework (FWR), a variable heavy chain domain (VH), or a variable light chain domain (VL) of the ABM expressed by the immune cell. In one embodiment, an amino acid sequence of the ABM expressed by the immune cell is determined from the determined nucleic acid sequence.

In one embodiment, the partition is a droplet, well or microwell.

In one embodiment, the known binding characteristic comprises a known binding affinity, a known on-rate, and/or a known off-rate.

In one embodiment, the reporter oligonucleotide(s) and/or nucleic acid barcode molecule(s) comprise a unique molecule identifier (UMI).

The present disclosure also provides a system for characterizing an ABM, the system comprising: (a) a first pre-determined antigen binding molecule (PABM)-associated bead, wherein the first PABM-associated bead comprises a first PABM, and a first PABM reporter oligonucleotide (b) a second pre-determined antigen binding molecule (PABM)-associated bead, wherein the second PABM-associated bead comprises a second PABM, and a second PABM reporter oligonucleotide (c) a target antigen. In one embodiment, the system further comprises a plurality of nucleic acid barcode molecules comprising a partition-specific barcode sequence and a capture sequence. In one embodiment, the system further comprises a partitioning system for generating a partition. In one embodiment, the partitioning system is a microfluidic device.

The present disclosure also provides a kit comprising: (a) a plurality of pre-determined antigen binding molecule (PABM)-associated beads, each comprising a PABM barcode sequence and (b) instructions for use

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a microfluidic channel structure for partitioning individual analyte carriers.

FIG. 2 shows an example of a microfluidic channel structure for the controlled partitioning of beads into discrete droplets.

FIG. 3 illustrates an example of a barcode carrying bead.

FIG. 4 illustrates another example of a barcode carrying bead.

FIG. 5 schematically illustrates an example microwell array.

FIG. 6 schematically illustrates an example workflow for processing nucleic acid molecules.

FIG. 7 schematically illustrates example labelling agents with nucleic acid molecules attached thereto.

FIG. 8A schematically shows an example of labelling agents. FIG. 8B schematically shows another example workflow for processing nucleic acid molecules. FIG. 8C schematically shows another example workflow for processing nucleic acid molecules.

FIG. 9 schematically shows another example of a barcode-carrying bead.

FIG. 10 shows an exemplary microfluidic channel structure for delivering barcode carrying beads to droplets.

FIG. 11 shows an exemplary embodiment of a method disclosed herein.

FIG. 12 shows an exemplary count matrix of a method disclosed herein.

FIG. 13 shows an exemplary graph showing a correlation between known binding characteristics of PABMs with their associated quantities of antigen barcode sequences.

### DETAILED DESCRIPTION OF THE DISCLOSURE

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The terms “a,” “an,” and “the,” as used herein, generally refers to singular and plural references unless the context clearly dictates otherwise. “A and/or B” is used herein to include all of the following alternatives: “A”, “B”, “A or B”, and “A and B”.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value±up to 10%, up to ±5%, or up to ±1%.

Headings, e.g., (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses). The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the term “barcoded nucleic acid molecule” and “barcoded polynucleotide” are generally used interchangeably herein to refer to a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence or a non-targeted sequence. The nucleic acid barcode molecule may be coupled to or attached to the nucleic acid molecule comprising the nucleic acid sequence. For example, a nucleic acid barcode molecule described herein may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcode molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcode molecule to generate the barcoded nucleic acid molecule, or the nucleic acid molecule comprising the nucleic acid sequence may be attached to the nucleic acid barcode molecule and subjected to a nucleic acid reaction (e.g., extension, ligation) to generate the barcoded nucleic acid molecule. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule (e.g., mRNA).

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a check swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle” may be used herein to generally refer to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a nucleus of a cell. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition can be a physical container, compartment, or vessel, such as a droplet, a flowcell, a reaction chamber, a reaction compartment, a tube, a well, or a microwell. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

The term “percent identity,” as used herein in the context of two or more proteins, refers to two or more sequences or subsequences that are the same or have a specified percentage of amino acids that are the same, e.g., about 70%, 72%, 74%, 75%, 76%, 79%, 80%, 81%, 82%, 83%, 84%, 85%, 86%, 87%, 88%, 89%, 90%, 91%, 92%, 93%, 94%, 95%, 96%, 97%, 98%, 9%9, 99.5%, 99.9%, or higher identity over a specified region, when compared and aligned for maximum correspondence over a comparison window or designated region as measured using a BLAST or BLAST 2.0 sequence comparison algorithms with default parameters described below, or by manual alignment and visual inspection. See, e.g., the NCBI web site at ncbi.nlm.nih.gov/BLAST. In some embodiments, this definition includes sequence comparison performed by a BLAST algorithm wherein the parameters of the algorithm are selected to give the largest match between the respective sequences over the entire length of the respective reference sequences. In some embodiments, this definition also includes sequences that have modifications such as deletions and/or additions (e.g., insertions), as well as those that have substitutions. Such modifications can occur naturally or synthetically. In some embodiments, sequence identity can be calculated over a region that is at least about 20 amino acids in length, or over a region that is 10-100 amino acids in length, or over the entire length of a given sequence. Sequence identity can be calculated using published techniques and widely available computer programs, such as the GCS program package (Devereux et al, Nucleic Acids Res (1984) 12:387), BLASTP, BLASTN, FASTA (Atschul et al., J Mol Biol (1990) 215:403). In some embodiments, sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof. Additional methodologies that can suitably be utilized to determine structural similarity or identity amino acid sequences include those relying on position-specific structure-scoring matrix (P3SM) that incorporates structure-prediction scores from Rosetta, as well as those based on a length-normalized edit distance as described previously in, e.g., Setcliff et al., Cell Host & Microbe 23(6), May 2018.

### Overview

Provided herein are methods, systems and kits for the determination of binding characteristics of ABMs, e.g., antibodies or antigen-binding fragments of antibodies, produced by immune cells, e.g., B cells, using single-cell immune profiling technologies. The methods, systems and kits provided herein employ new and useful reagents that improve the ability to infer a binding characteristic, e.g., binding affinity, of antigen and its antigen receptor. Using single-cell technology, a UMI count for a particular antigen should be a function of the affinity (and other kinetic properties) of e.g., a BCR-antigen interaction. However, the affinity is hard to infer given that the UMI-affinity relationship is not direct and is affected by multiple biological and experimental factors. In the case of some reagents, further described herein below, the reagents offer the ability to better infer binding characteristics ABMs that bind a target antigen-containing reagent with greater accuracy, thus saving resources that would have been spent identifying and characterizing ABMs that may not bind the reagent with acceptable e.g., binding affinity. The ability to efficiently and cost-effectively identify and characterize ABMs is of value for the development of new and useful immunotherapeutics for treatment of disease, e.g., cancer or infection.

### Methods of the Disclosure

As described in more detail below, one aspect of the disclosure relates to new approaches and methods for the determination of binding characteristics of antigen-binding molecules (ABMs), e.g., antibodies (Abs) or antigen-binding fragments of antibodies using pre-determined antigen binding molecule (PABM)-associated beads. The methods, systems and kits provided herein may also characterize an ABM by identifying it as having a particular nucleic acid sequence(s) and/or as having particular amino acid sequence(s).

The present disclosure provides a method that involves partitioning a reaction mixture, or a portion thereof, into a plurality of partitions. The reaction mixture includes a plurality of pre-determined antigen binding molecule (PABM)-associated beads. A first PABM-associated bead of the plurality of PABM-associated beads includes a first PABM, and a first PABM reporter oligonucleotide comprising a first PABM barcode sequence identifying the first PABM. The reaction mixture also includes a plurality of antigens comprising a first antigen operatively coupled to a first antigen reporter oligonucleotide comprising a first antigen reporter barcode sequence, wherein the first antigen binds to the first PABM of the PABM-associated bead. Partitioning the reaction mixture provides (i) a first partition comprising the first PABM-associated bead, (ii) the first antigen, and (iii) a plurality of first partition nucleic acid barcode molecules comprising a first partition-specific barcode sequence. The method then involves generating (i) a first barcoded polynucleotide comprising (i) the first partition barcode sequence or a reverse complement thereof and (ii) the first antigen reporter barcode sequence or reverse complement thereof; and (ii) a second barcoded polynucleotide comprising (i) the first partition barcode sequence or reverse complement thereof and (ii) the first PABM reporter barcode sequence or reverse complement thereof.

The PABM and ABMs, as provided herein, may be an antibody, or an antigen-binding fragment thereof. The PABM or ABM may be an antibody having an Immunoglobulin (Ig) A (e.g., IgA1 or IgA2), IgD, IgE, IgG (e.g., IgG1, IgG2, IgG3 and IgG4) or IgM constant region. The PABM or ABM may be a fragment of the antibody, that may be any naturally occurring, enzymatically obtainable, synthetic, or genetically engineered polypeptide or glycoprotein that specifically binds an antigen to form a complex. A PABM or ABM that is a fragment of an antibody may be one of: (i) Fab fragments; (ii) F(ab′)2 fragments; (iii) Fd fragments; (iv) Fv fragments; (v) single-chain Fv (scFv) molecules; (vi) sdAb fragments; and (vii) minimal recognition units consisting of the amino acid residues that mimic the hypervariable region of an antibody (e.g., an isolated complementarity determining region (CDR) such as a CDR3 peptide), or a constrained FWR3-CDR3-FWR4 peptide. Further, an antigen-binding fragment of an antibody may be an engineered molecule, such as a domain-specific antibody, single domain antibody, chimeric antibody, CDR-grafted antibody, diabody, triabody, tetrabody, minibody, nanobody (e.g., monovalent nanobodies, bivalent nanobodies, etc.), a small modular immunopharmaceutical (SMIP), or a shark immunoglobulin new antigen receptor (IgNAR) variable domain.

The first PABM, used in the methods provided herein, may be an ABM that binds to the first antigen. The first PABM may be known to bind to the first antigen. Accordingly, the first antigen may be any antigen to which the PABM is known to bind.

In the methods provided herein, a reaction mixture, or a portion thereof, may be partitioned into a plurality of partitions. The reaction mixture, or portion thereof, for partitioning in the methods may include a plurality of PABM-associated beads.

A PABM-associated may be any bead, as described herein, attached to a PABM. The PABM-associated bead may be porous, non-porous, solid, semi-solid, semi-fluidic, fluidic, and/or a combination thereof.

In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A PABM-associated bead may be of any suitable shape. Examples of bead shapes include, but are not limited to, spherical, non-spherical, oval, oblong, amorphous, circular, cylindrical, and variations thereof.

The PABMs may be attached directly or indirectly to the bead. In some cases, the PABMs may be covalently linked to the bead. In some cases, the PABMs are covalently linked to the bead via a linker.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. Exemplary precursors comprising functional groups are described in PCT/US2014/044398, which is hereby incorporated by reference in its entirety.

The reaction mixture, or portion thereof, that may be partitioned into the plurality of partitions in the methods provided herein, may include a plurality of antigens in addition to the PABM-associated beads. The plurality of antigens may include a first antigen that binds to a first PABM of the PABM-associated bead.

The partitioning of this reaction mixture, or a portion thereof, may provide a first partition of a plurality of partitions that includes (i) a first partition comprising the first PABM-associated bead, (ii) the first antigen.

The reaction mixture, or portion thereof, that may be partitioned into the plurality of partitions in the methods provided herein, may further include a plurality of additional labelling agents. In some embodiments, one or more of the additional labelling agents comprise a detectable label, e.g., a detectable label described herein. In some embodiments, one or more of the additional labelling agents comprise a reporter oligonucleotide. In some embodiments, reporter oligonucleotides of the one or more additional labeling agents have different primer sequences, e.g., different sequencing primer sequences than reporter oligonucleotides coupled to the target MHC antigens.

In any of the methods provided herein, the provided partition including the first PABM-associated bead, (ii) the first antigen, may further include a plurality of nucleic acid barcode molecules. A nucleic acid barcode molecule of the plurality may have a partition-specific barcode sequence.

In the methods provided herein, barcoded nucleic acid molecules may be generated. In some embodiments, the barcoded nucleic acid molecules may be generated in the partition.

The generated barcoded nucleic acid molecules may include a first barcoded nucleic molecule/polynucleotide that includes: (i) the first partition barcode sequence or a reverse complement thereof and (ii) the first antigen reporter barcode sequence or reverse complement thereof; and (ii) a second barcoded polynucleotide comprising (i) the first partition barcode sequence or reverse complement thereof and (ii) the first PABM reporter barcode sequence or reverse complement thereof.

In some embodiments of the methods provided herein, the reaction mixture, or portion thereof, for partitioning in the methods may also include a plurality of immune cells. The plurality of immune cells may be a plurality of B cells, a plurality of T cells, or a plurality of B and T cells. In instances in which the reaction mixture includes a plurality of B cells, a plurality of T cells, or a plurality of B cell and T cells, the plurality of immune cells may be have been enriched from a sample prior to inclusion in the reaction mixture for the partitioning. The enrichment for B cells, T cells or B and T cells for inclusion in the reaction mixture may be performed by any method known in the art, such as by labeling cells with a detectable moiety, e.g., fluorescent or magnetic marker (e.g., based on an expressed cell surface marker or another marker) and subjected to a FACS or MACS process to separate the B, T or T and T cells from other cells. In some embodiments, the expressed cell surface marker for enriching for B cells may be CD19. In other embodiments, the expressed cell surface marker for enriching for T cells may be CD3, CD4 and/or CD8.

The plurality of immune cells for inclusion in the reaction mixture, whether or not enriched for B, T or B and T cells, may be from a sample of a subject, e.g., a vertebrate non-mammalian subject or mammalian subject, e.g., a human or mouse (e.g., transgenic mouse). The sample of the subject may have been obtained by biopsy, core biopsy, needle aspirate, or fine needle aspirate. The plurality of immune cells for inclusion in the reaction mixture may be from a fluid sample of the subject, such as a blood sample. The sample may have been processed prior to its inclusion in the reaction mixture. The processing of the sample may include steps such as filtration, selective precipitation, purification, centrifugation, agitation, heating, and/or other processes. For example, a sample may be filtered to remove a contaminant or other materials. In some cases, cells and/or cellular constituents of a sample may be processed to separate and/or sort cells of different types, e.g., to separate B cells, as discussed herein (e.g., by FACS or MACS based on an expressed cell surface marker), from other cell types. A separation process may be a positive selection process, a negative selection process (e.g., removal of one or more cell types and retention of one or more other cell types of interest), and/or a depletion process (e.g., removal of a single cell type from a sample, such as removal of red blood cells from peripheral blood mononuclear cells). The subject, from whom the sample may have been obtained, may have been exposed to, expected to have been exposed, resistant to, or suspected to be resistant to, or immunized against the target antigen.

The partitioning of this reaction mixture, or a portion thereof, may provide a second partition of a plurality of partitions that includes (i) an immune cell expressing an antigen binding molecule (ABM), (ii) a target antigen operatively coupled to a target antigen reporter oligonucleotide comprising a target antigen barcode sequence, and (iii) a plurality of second partition nucleic acid barcode molecules comprising a second partition-specific barcode sequence.

The first antigen and/or the target antigen may be an antigen associated with an infectious agent, such as a viral, bacterial, parasitic, protozoal or prion agent. A first antigen and/or the target antigen associated with an infectious agent, wherein the infectious agent is a viral agent, may be an antigen of an influenza virus, a coronavirus, a retrovirus, a rhinovirus, or a sarcoma virus. It may be an antigen associated with a severe acute respiratory syndrome coronavirus 1 (SARS-COV-1), a SARS-COV-2, a Middle East respiratory syndrome coronavirus (MERS-COV)), or human immunodeficiency virus (HIV), influenza, respiratory syncytial virus, or Ebola virus. Example first antigens and/or the target antigens of viral agents include corona virus spike(S) protein, influenza hemagglutinin protein, an HIV envelope protein or any other a viral glycoprotein. Other first antigens and/or the target antigens may be antigens associated with a tumor or a cancer. First antigen and/or the target antigen associated with a tumor or cancer may be, for example, epidermal growth factor receptor (EGFR), CD38, platelet-derived growth factor receptor (PDGFR) alpha, insulin growth factor receptor (IGFR), CD20, CD19, CD47, or human epidermal growth factor receptor 2 (HER2). Other first antigens and/or the target antigens associated with a tumor or cancer may be checkpoint molecules. Check point molecules that may be the first antigen and/or the target antigen include, but are not limited to, CD38, PD-1, CTLA-4, TIGIT, LAG-3, VISTA, TIM-3. Further first antigens and/or the target antigens may be cytokines, GPCRs, cell-based co-stimulatory molecules, cell-based co-inhibitory molecules, ion channels, and growth factors. Further still, first antigens and/or the target antigens may be associated with a degenerative condition or disease (e.g., an amyloid protein).

In some embodiments, the first antigen and/or the target antigen, for binding to the PABM and/or ABM may be of a length of at least 20 amino acid residues, at least 40 amino acid residues, at least 60 amino acid residues, at least 80 amino acid residues, at least 100 amino acid residues, at least 200 amino acid residues, at least 300 amino acid residues, at least 400 amino acid residues, at least 500 amino acid residues, at least 600 amino acid residues, at least 700 amino acids, at least 800 amino acid residues, at least 900 amino acid residues, at least 1000 amino acid residues, at least 1100 amino acid residues, at least 1200 amino acid residues, at least 1300 amino acid residues, up to 40 amino acid residues, up to 60 amino acid residues, up to 80 amino acid residues, up to 100 amino acid residues, up to 200 amino acid residues, up to 300 amino acid residues, up to 400 amino acid residues, up to 500 amino acid residues, up to 600 amino acid residues, up to 700 amino acids, up to 800 amino acid residues, up to 900 amino acid residues, up to 1000 amino acid residues, up to 1100 amino acid residues, up to 1200 amino acid residues, or up to 1300 amino acid residues. The first antigen and/or the target antigen may be an antigen that includes one domain, at least one domain, two domains, at least two domains, three domains, at least three domains, four domains, at least four domains, five domains, at least five domains, six domains, at least six domains, seven domains, at least seven domains, eight domains, at least eight domains, nine domains, at least nine domains, ten domains, at least ten domains, at least thirty domains, at least forty domains, at least fifty domains, at least sixty domains, at least seventy domains, at least eighty domains, at least ninety domains or at least one hundred domains. The first antigen and/or the target antigen may be an antigen that includes at most two hundred domains, at most 175 domains, at most 150 domains, at most 125 domains, at most 100 domains, at most 75 domains, at most 50 domains, at most 25 domains, at most 20 domains, at most 15 domains, at most 10 domains, or at most 5 domains.

In further embodiments, the first antigen and/or the target antigen may be a protein or peptide as expressed by a cell, e.g., full-length antigen that may or may not include its leader sequence and may or may not have undergone a similar cell-processing step.

In other embodiments, the first antigen and/or the target antigen includes a MHC molecule bound to an antigenic peptide.

The antigenic peptide may be a peptide, or peptide fragment, of any target antigen to which binding by an ABM is desirable. The antigenic peptide may be a peptide, or peptide fragment of a target antigen that may be associated with an infectious agent, such as a viral, bacterial, parasitic, protozoal or prion agent. It may be a peptide or peptide fragment of a target antigen associated with a tumor or a cancer, e.g., growth factor receptor or transcription factor. Further, it may be a peptide or peptide fragment of a target antigen associated with a degenerative condition or disease.

In some embodiments wherein the first antigen and/or the target antigen comprises an MHC molecule, the MHC molecule may include a MHC class I molecule described in the European Patent Publication No. EP 385722 A1. In some embodiments, the MHC monomers may include a MHC class I molecule comprising a heavy chain comprising an alpha-1 domain and an alpha-2 domain connected by a disulfide bridge, wherein the heavy chain includes the amino acid sequence of SEQ ID NO: 1 described in EP 385722 A1. In some embodiments, the heavy chain includes an amino acid sequence having at least 80% sequence identity to SEQ ID NO: 1 described in EP 385722 A1. In some embodiments, the MHC monomers may include a MHC class I molecule comprising a heavy chain comprising an alpha-1 domain and an alpha-2 domain connected by a disulfide bridge, wherein the heavy chain comprising an amino acid sequence selected from (a) SEQ ID NO: 1 described in EP385722A1, or (b) an amino acid sequence having at least 80% identity to (a) wherein a mutant cysteine residue is positioned in the alpha-1 domain at amino acid residue 84 or 85 and a mutant cysteine residue is positioned in the alpha-2 domain at amino acid residue 139.

In one embodiment, the target antigen is different from the first antigen.

In one embodiment, the target antigen is the same as the first antigen.

The reaction mixture, or portion thereof, that may be partitioned into the plurality of partitions in the methods provided herein, may further include a plurality of additional labelling agents. In some embodiments, the additional labeling agents are configured to bind or otherwise couple to one or more cell-surface features of an immune cell. In some embodiments, such additional labeling agents can be used to characterize cells and/or cell features. In some embodiments, one or more of the additional labelling agents comprise a detectable label, e.g., a detectable label described herein. In some embodiments, one or more of the additional labelling agents comprise a reporter oligonucleotide. In some embodiments, reporter oligonucleotides of the one or more additional labeling agents have different primer sequences, e.g., different sequencing primer sequences than reporter oligonucleotides coupled to the target MHC antigens. In some embodiments, the immune cells are contacted with the target antigen, then with the additional labelling agents.

As described above, in any of the methods provided herein, the provided partition including the immune, e.g., B or T cell, bound to the target antigen, may further include a plurality of nucleic acid barcode molecules. A nucleic acid barcode molecule of the plurality may have a partition-specific barcode sequence and may further have a capture sequence. In some embodiments, the capture sequence may be configured to couple to an mRNA or DNA analyte of the immune cell, e.g., B cell, in the provided partition. A capture sequence configured to couple to an mRNA or DNA analyte of an immune cell may include a polyT sequence or be complementary to a gene-specific sequence, e.g., sequence of an immunoglobulin variable or constant region or B cell receptor variable or constant region. Alternatively, the capture sequence may be configured to couple to non-templated nucleotides appended to a cDNA reverse transcribed, by a reverse transcriptase having terminal transferase activity, from an mRNA analyte of the immune cell, e.g. B cell, in the provided partition. In embodiments in which the capture sequence is configured to couple to non-templated nucleotides appended to a cDNA reversed transcribed from the mRNA analyte, the mRNA analyte may be reversed transcribed to the cDNA using a polyT primer or a primer with a sequence complementary to a gene-specific sequence as discussed above. During reverse transcription, the reverse transcriptase, via its terminal transferase activity, may append one or more non-templated nucleotides, e.g., cytosines, to the cDNA. If the non-templated nucleotides appended to the cDNA are cytosines, the capture sequence of the nucleic acid barcode molecule may include one or more guanines. The nucleic acid barcode molecules, in addition to the partition-specific barcode sequence and capture sequence, may further include one or more functional sequences, such as a unique molecule identifier (UMI), sequencer attachment sequence, sequencing primer sequence, amplification primer sequence, or the complements thereof.

In the methods provided herein, third and fourth barcoded nucleic acid molecules may be generated. In some embodiments, the barcoded nucleic acid molecules are generated in the partition.

The generated third and fourth barcoded nucleic acid molecules include a barcoded nucleic molecule that includes: (i) the second partition barcode sequence or a reverse complement thereof and (ii) the target antigen reporter barcode sequence or a reverse complement thereof, and (iv) a fourth barcoded polynucleotide comprising (i) the second partition barcode sequence or a reverse complement thereof and (ii) a first nucleic acid sequence encoding at least a portion of the ABM expressed by the immune cell, or a reverse complement thereof. This generated barcoded nucleic acid molecule may characterize the ABM. In instances in which the immune cell in the provided partition was a B cell, the generated barcoded nucleic acid molecule may characterize a B cell receptor, an antibody or an antigen-binding fragment of an antibody expressed by the B cell that had been in the provided partition. It may characterize the ABM by identifying the ABM. The ABM may been characterized, e.g., identified, based on the generated barcoded nucleic acid molecule having been subject to a step of sequencing, e.g., by having determined a sequence of the ABM based on the generated barcoded nucleic acid molecule. The determined sequence of the ABM may be a nucleic acid sequence encoding the ABM, e.g., antibody or antigen-binding fragment of the antibody, or an amino acid sequence of the ABM. The nucleic acid and/or amino acid sequence need not be full length full length sequence of the ABM.

In some embodiments, the plurality of PABM associated beads includes a second PABM-associated bead. In such embodiments, the second PABM-associated bead may include a second PABM and a second PABM reporter oligonucleotide comprising a second PABM barcode sequence identifying the second PABM. The partitioning of this reaction mixture, or a portion thereof, may provide a first partition of a plurality of partitions that includes (i) a 3^{rd} partition comprising the second PABM-associated bead, and (ii) a plurality of third partition nucleic acid barcode molecules comprising a third partition-specific barcode sequence. In such embodiments, fifth barcoded nucleic acid molecules may be generated. In some embodiments, the fifth barcoded nucleic acid molecules may be generated in the partition. The generated fifth barcoded nucleic acid molecules may include a first barcoded nucleic molecule/polynucleotide that includes: a 5th barcoded polynucleotide comprising the 3^{rd} partition barcode sequence or reverse complement thereof and the 2^{nd} PABM reporter barcode sequence or reverse complement thereof.

In some embodiments, the second PABM binds the first antigen. Thus, in these embodiments, the third partition may further include the first antigen operatively coupled to the first antigen reporter oligonucleotide. The method may then involve generating a 6th barcoded polynucleotide that includes the 3^{rd} partition barcode sequence or reverse complement thereof and the first antigen reporter barcode sequence or reverse complement thereof.

In some embodiments, the second PABM binds a second antigen. Thus, in these embodiments, the third partition may further include the second antigen operatively coupled to a second antigen reporter oligonucleotide comprising a second antigen reporter barcode sequence. The method may then involve generating a 7th barcoded polynucleotide that includes the 3^{rd} partition barcode sequence or reverse complement thereof and the second antigen reporter barcode sequence or reverse complement thereof.

In examples in which the plurality of antigens include a target antigen that is coupled to a reporter oligonucleotide, the reporter oligonucleotide that may be coupled to the target antigen, may include a reporter barcode sequence. The reporter barcode sequence of the reporter oligonucleotide may identify the antigen, e.g., target or non-target antigen, to which it is coupled. Thus, a reporter oligonucleotide coupled to the target antigen, may include a reporter barcode sequence that identifies the target antigen. In addition to including a reporter barcode sequence, the reporter oligonucleotide, may include a capture handle sequence and may, optionally, additionally include functional sequences such as a UMI or primer binding sequence. The capture handle sequence of the reporter oligonucleotide may be configured to couple to a capture sequence of one or more additional nucleic acid barcode molecules of the plurality of nucleic acid barcode molecules, e.g., plurality of nucleic acid barcode molecules in the provided partition with the immune cell bound to the target antigen. In these embodiments, the additional generated barcoded nucleic acid molecule may include a sequence of the reporter oligonucleotide, e.g, reporter barcode sequence that identifies the target antigen bound by the immune cell in the provided partition, or a reverse complement thereof, and the partition-specific barcode sequence or a reverse complement thereof.

This additional generated barcoded nucleic acid molecule may characterize the ABM, e.g., antibody or antigen-binding fragment of the antibody, expressed by an immune, e.g., B, cell. The additional generated barcoded nucleic acid molecule, or amplicon or derivative thereof, may further be sequenced. Sequencing of the additional barcoded nucleic acid molecule or amplicon or derivative thereof, e.g., determining sequence of the additional barcoded nucleic acid molecule, may characterize the ABM, e.g., antibody or antigen-binding fragment of an antibody, expressed by the immune cell, e.g., B cell, in the provided partition by confirming its binding to, and/or as having affinity for, the target antigen.

The sequencing of any of the generated barcoded nucleic acid molecules may be performed by any of a variety of approaches, systems, or techniques, including next-generation sequencing (NGS) methods. Sequencing may be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification. Non-limiting examples of nucleic acid sequencing methods include Maxam-Gilbert sequencing and chain-termination methods, de novo sequencing methods including shotgun sequencing and bridge PCR, next-generation methods including Polony sequencing, 454 pyrosequencing, Illumina sequencing, SOLiD™ sequencing, Ion Torrent semiconductor sequencing, HeliScope single molecule sequencing, nanopore sequencing (Oxford Nanopore) and SMRT® sequencing.

Further, sequence analysis of the barcoded nucleic acid molecules may be direct or indirect. Thus, the sequence analysis can be performed on a barcoded nucleic acid molecule or it can be a molecule which is derived therefrom (e.g., a complement or amplicon thereof).

Other examples of methods for sequencing the barcoded nucleic acid molecules include, but are not limited to, DNA hybridization methods, restriction enzyme digestion methods, Sanger sequencing methods, ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, Solexa Genome Analyzer sequencing, MS-PET sequencing, whole transcriptome sequencing, and any combinations thereof.

In some embodiments, the first PABM binds to the first antigen with a known binding characteristic. In some embodiments, the known binding characteristic includes a known binding affinity, a known on-rate, and/or a known off-rate. In some embodiments, the known binding characteristic of the first PABM for the first antigen was determined a priori, e.g., based on prior experimentation.

The prior experimentation may include determination of binding affinity, on-rates, and/or off-rates. Binding affinity between two molecules can be quantified by determination of the equilibrium dissociation constant (K_{D}). In turn, K_{D} can be determined by measurement of the kinetics of complex formation and dissociation using, e.g., the surface plasmon resonance (SPR) method (Biacore™). The rate constants corresponding to the association (e.g., on-rate) and the dissociation (e.g., off rate) of a monovalent complex are referred to as the association rate constants kₐ (or kₒₙ) and dissociation rate constant k_{d} (or k_{off}), respectively. K_{D} is related to kₐ and k_{d} through the equation K_{D}=k_{d}/kₐ. The value of the dissociation constant can be determined directly by various methods, and can be computed even for complex mixtures by methods such as those set forth in Caceci et al. (1984, Byte 9:340-362). For example, the K_{D} can be established using a double-filter nitrocellulose filter binding assay such as that disclosed by Wong & Lohman (1993, Proc. Natl. Acad. Sci. USA 90:5428-5432). Other methods to evaluate the binding ability of the antibodies and antigen-binding fragments include, for example, ELISAs, Western blots, RIAs, Octet® HTX biosensor, solution-affinity ELISA, and flow cytometry analysis. The binding characteristics of PABMs for their antigens (e.g., first antigen) can be determined a priori by, for example, by Surface Plasmon Resonance (SPR), e.g. by using a Biacore™ system, or KinExA. The binding characteristics of PABMs for their antigens (e.g., first antigen) can be determined a priori by, for other example, by the Scatchard method described by Frankel et al., Mol. Immunol, 16:101-106, 1979.

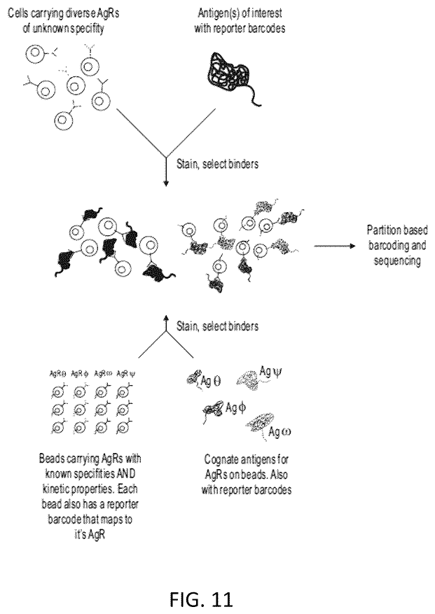

FIG. 11 depicts an exemplary embodiment of a method disclosed herein. In some embodiments, a reaction mixture is prepared by incubating beads carrying PABMs that bind to known antigens (e.g., a first antigen as described herein). While FIG. 11 depicts the known antigens as Agθ, AgΦ, Agψ, and Agω, and their corresponding known PABMs as AgRθ, AgRΦ, AgRψ, and AgRω, it is to be understood that the PABMs may include antigen-binding fragments or antigen binding domains of full antigen receptors as opposed to the full antigen receptors themselves, and it is also to be understood that while four different PABMs and four different corresponding antigens are depicted, methods disclosed herein may utilize, e.g., one PABM that binds to a first antigen (for example with a known binding characteristic), one PABM that binds to two different known antigens with two different known binding characteristics (e.g., two different K_{D}·s, or two different kₒₙ or k_{off} rates), or two or more PABMs that bind to a known antigen (e.g., a first antigen) with two different known binding characteristics. As shown in FIG. 11, the reaction mixture may further comprise immune cells carrying diverse ABMs (e.g., diverse antigen receptors) and a target antigen (“Ag1”) operatively coupled with a reporter oligonucleotide comprising a target antigen reporter barcode sequence.

As shown in FIG. 11, after the PABM-carrying beads and immune cells are incubated with the target and known antigens, a selection step may be carried out. In some embodiments wherein the target antigens and/or the known antigens are coupled with detectable labels (e.g., fluorophores) in addition to reporter oligonucleotides, the selection step may include selecting based on detection of the detectable labels (e.g., by FACS).

As shown in FIG. 11, the reaction mixture, (or portion of the reaction mixture as selected, e.g., by FACS) is subjected to partition-based barcoding and sequence analysis, according to any one of the partitioning methods disclosed herein. Such partition-based barcoding and sequence analysis may be used to associate sequence reads from barcoded polynucleotides with partition barcode sequences.

In some embodiments, the method further comprises generating a count matrix. The count matrix may include an association of partition barcode sequences with first PABM barcode sequences and a quantity of first antigen barcode sequences. In some embodiments, the association of partition barcode sequences with first PABM barcode sequences and a quantity of first antigen barcode sequences may indicate one or more first antigens have been bound by the first PABM. In some embodiments, the association of partition barcode sequences with first PABM barcode sequences and a quantity of first antigen barcode sequences may indicate that none of the first antigens have bound the first PABM because, for example, either (i) the PABM barcode sequence was absent from the partition or (ii) the first antigen barcode sequence was absent from the partition. The count matrix may use, for example, a quantity/number of UMIs, of generated first antigen barcode sequences. For example, the count matrix may involve quantity/number of first antigen UMIs associated with the first PABM, e.g., quantity/number of first antigen UMIs associated with the same partition-specific barcode as the first PABM.

In some embodiments, the quantity of first antigen barcode sequences and/or first PABM barcode sequences may be determined based on quantity of sequence reads and amplification products such as RCA or PCR amplification products comprising such barcode sequences.

In some embodiments, the count matrix may be used to correlate the known binding characteristic of the first PABM for the first antigen with the quantity of first antigen barcode sequences.

In some embodiments, the second PABM binds to the first antigen or the second antigen with a known binding characteristic. The known binding characteristic of the second PABM for the first antigen or the second antigen may be different from that of the first PABM for the first antigen. For example, in some embodiments wherein the first PABM binds to the first antigen with a known binding affinity (e.g., a known K_{D} value), the second PABM binds to the first antigen or a second antigen with a different known binding affinity (e.g., a known KD value that different from the K_{D} value of the first PABM). In certain embodiments, there may be a 0.5-fold, a one-fold, a two-fold, a five-fold, a ten-fold, or a greater than ten-fold difference between the known binding affinity of the first PABM for the first antigen and the known binding affinity of the second PABM for the first or second antigen.

It is to be understood that any number of PABMs and cognate PABM antigens may be used in the methods disclosed herein. In some embodiments, the additional PABMs may bind to their cognate PABM antigens with different known binding characteristics. For example, third PABMs may bind to their cognate antigen with a known binding affinity that is different from the known binding affinity of the first PABM for the first PABM antigen and different from the known binding affinity of the second PABM for the first or second PABM antigen. In such embodiments, the PABMs may represent a range of known binding characteristics (e.g., may represent a range of known binding affinities) for their cognate antigens. By way of example only, the PABMs may represent binding affinities (e.g., K_{D} values) for their cognate antigen that range from the mM to pM range, the μM to pM range, the nM to pM range, and the like.

In some embodiments, a user may wish to have greater representation of known binding characteristics (e.g., binding affinities) across a subrange, and may select the subrange for greater representation, e.g., by selecting PABMs within the subrange that have smaller differences in their known binding characteristics relative to the fold-differences in known binding characteristics that fall outside the subrange. By way of example only, a user who wishes to include PABMs that represent a range of K_{D} values in the mM to pM range, with greater representation in the nM to pM subrange, may utilize PABMs with, e.g., 2-fold differences in K_{D} value across the nM to pM subrange and 10-fold differences in K_{D} value across the mM to nM range.

In some embodiments, the count matrix further includes an association of partition barcode sequences with second PABM barcode sequences and quantity of first or second antigen barcode sequences or (ii) the method further comprises generating a count matrix that includes an association of partition barcode sequences with second PABM barcode sequences and quantity of first or second antigen barcode sequences.

The count matrix may further include an association of partition barcode sequences with target antigen UMI counts and nucleic acid sequences encoding the ABM expressed by the immune cell. Alternatively, the method described herein may further include generating a count matrix that includes an association of partition barcode sequences with target antigen UMI counts and nucleic acid sequences encoding the ABM expressed by the immune cell.

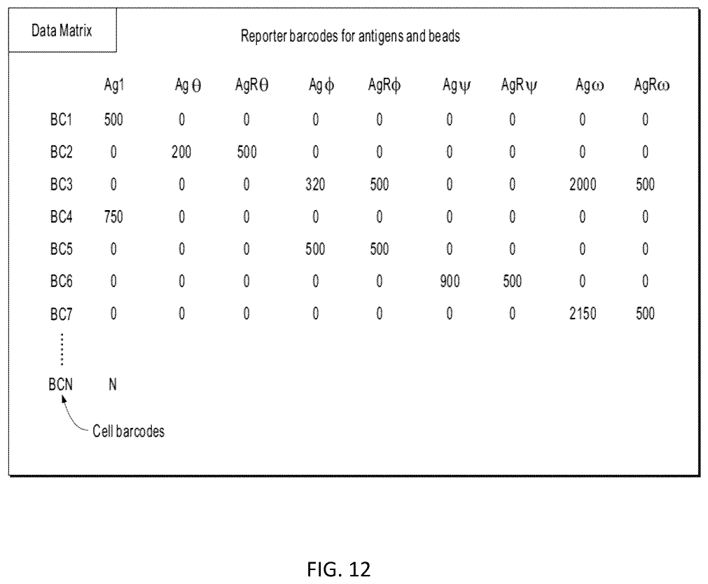

FIG. 12 depicts an exemplary count matrix of a method disclosed herein. As shown in FIG. 12, the count matrix may include one or more reporter barcode UMI counts for a target antigen (e.g., Ag1), and pairs of PABM-associated beads and their cognate antigens (e.g., Agθ and AgRθ, etc). For example, as shown in FIG. 12, each row of the count matrix may correspond to one or more (e.g., BC1, . . . , BCN) cell barcodes while each column of the count matrix may correspond to one target antigen and a quantity of pairs of PABM-associated bead and its cognate antigen (e.g., Agθ and AgRθ, etc). Accordingly, each count (e.g. 500, 200, 320, etc) of the count matrix may correspond to the e.g., affinity of the PABMs for its cognate antigen.

In some embodiments, the count matrix may be used to correlate the known binding characteristic of the second PABM for the first antigen or the second antigen with the quantity of first antigen or second antigen barcode sequences.

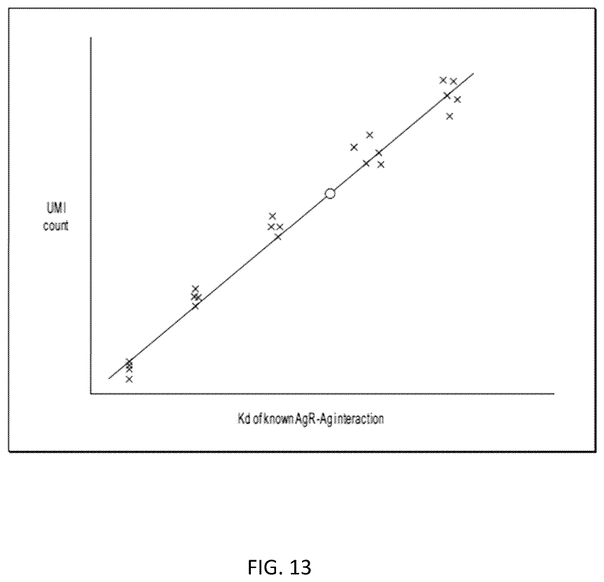

A correlation may be made between the known binding characteristics of the 1^{st} and 2^{nd} PABMs with their associated quantities of 1^{st} or 2^{nd} antigen barcode sequences and this may be used to infer a relationship between antigen barcode sequence quantity and the binding characteristic. For example, FIG. 13 depicts an exemplary graph showing a correlation between known binding characteristics of PABMs with their associated quantities of antigen barcode sequences. As shown in FIG. 13, when the known binding characteristic of the PABMs with their cognate antigens is a Kd value, the known Kd value may be plotted on an x-axis. The corresponding UMI counts from the count matrix may be plotted on a y-axis. FIG. 13 shows the resulting correlation between the Kd of the PABMs and the UMI counts.

The inferred relationship may then be used to estimate the binding characteristic of the ABM for the target antigen. For example, as shown in FIG. 13, the UMI count for the target antigen (e.g., Ag1 in the count matrix), may be used to extrapolate the Kd value of its corresponding ABM.

It will be also be understood that in any of the methods provided herein, the plurality of antigens is not necessarily limited to including a target antigen. Rather, the plurality of antigens may additionally include further antigens. In some embodiments, further antigens may include one or more negative control antigens. Negative control antigens can include antigens to which ABMs are not expected to bind. In some embodiments, negative control antigens are selected such that they are not expected to bind to immune cells of the reaction mixture. For example, a negative control antigen may be an antigen for which a subject (e.g., a human subject) is not expected to develop an immune response. Such a non-target antigen may be an antigen endogenous to and abundantly expressed in a subject, e.g., a human subject, e.g., human serum albumin (HSA). In some embodiments, negative control antigens can be coupled to a fluorophore to allow for identification of non-specific ABMs that bind to labeled antigens by interacting with the fluorophore rather than with the antigen of interest. In some embodiments, a single negative control antigen, e.g., HSA, can be coupled to two different detectable labels such as phycoerythrin (PE) and allophycocyanin (APC) to generate two control reagents.

In some embodiments, negative control antigens can include a reporter oligonucleotide associated with any one or more of: a detectable label, a support, and a ligand with binding affinity for a binding region of the support. Exemplary detectable labels (e.g., fluorophores) are further disclosed herein. Exemplary supports (e.g., streptavidin, avidin) and their ligands (e.g., biotin) are also further disclosed herein. For example, a negative control antigen can be or include a complex comprising a detectable label, support, and/or ligand with binding affinity for a binding region of the support. For example, a negative control antigen can be or can include a biotin-saturated streptavidin comprising a detectable label and/or a reporter oligonucleotide. Such negative control antigens can be used to distinguish antibodies that specifically bind a target antigen from antibodies that non-specifically bind to any one or more of the detectable label (e.g., fluorophore), support (e.g., streptavidin, avidin), and ligand (e.g., biotin).

In some embodiments of the methods disclosed herein, negative control antigens can include a streptavidin molecule saturated with biotin, wherein the streptavidin is coupled to a reporter oligonucleotide. In some embodiments, negative control antigens can include (i) biotinylated human serum albumin complexed with a streptavidin, wherein the streptavidin is coupled to a reporter oligonucleotide.

In some embodiments wherein the first antigen and/or the target antigen comprises MHC, a negative control antigen comprises an MHC bound to a control peptide. In some embodiments, the control peptide may be a scrambled peptide, serum albumin peptide, a heteroclitic peptide, or peptide to which immune cells of the sample are naïve. The scrambled peptide may have the same amino acid residue composition as a target antigenic peptide (bound to the first MHC molecule of the target MHC molecule complex), wherein the amino acid residues are presented in a different, e.g., scrambled, order relative to that of the target antigenic peptide. The serum albumin peptide may be a human or mouse serum albumin peptide. The control peptide may be any peptide, e.g., not only a serum albumin peptide, to which the ABMs of the plurality of immune cells would not be expected to bind, e.g., cardiolipin, keyhole limpet hemocyanin, flagellin or insulin. In instances in which the control peptide is a peptide to which ABMs of the plurality of immune cells would not be expected to bind, the control peptide may be a peptide of an abundantly expressed self-antigen of a subject from which the plurality of immune cells had been obtained. In other instances in which the control peptide is a peptide to which ABMs of the plurality of immune cells would not be expected to bind, the control peptide may be a peptide or peptide fragment of an antigen to which the plurality of immune cells are naïve. For example, the control peptide may be a peptide or peptide fragment of an antigen of a virus, e.g. HIV (e.g., TPGPGVRYPL), if the subject from which the plurality of immune cells have been obtained, has not been exposed to the virus, e.g., HIV. For other example, the control peptide may be a heteroclitic peptide. Heteroclitic peptides may include peptides having valine, or leucine or other suitable residues at positions that anchor the peptide to the second MHC molecule, e.g., position 2 and/or a C-terminal residue, but alanine residues at the remaining amino acid positions (e.g., ALAAAAAAV, ATAAAAAAK, AYAAAAAAL, APAAAAAAV or RYAAAAALL). Additional examples of negative control peptides include ASYAAAAV and vaccinia virus peptide TSYKFESV.

Further antigens may include one or more additional target antigens. Further antigens may refer to the inclusion of one, at least one, two, at least two, three, at least three, four, at least four, five, at least five, six, at least six, seven, at least seven, eight, at least eight, nine, at least nine, ten, at least ten, twenty, at least twenty, thirty, at least thirty, forty, at least forty, fifty, at least fifty, sixty, at least sixty, seventy, at least seventy, eighty, at least eighty, ninety, at least ninety, one hundred, at least one hundred, five hundred, or at least five hundred additional antigens in the plurality of antigens.

In any of the methods described herein, it will be understood that the partitioning of the reaction mixture, or portion thereof, may partition more than one immune cell of the plurality of immune, e.g. B, cells into more than one of a plurality of partitions. The partitioning of the reaction mixture may partition a first immune cell of the plurality of immune cells into a first partition, it may further partition a second immune cell of the plurality of immune cells into a second partition. Moreover, it may additionally partition a third immune cell of the plurality of immune cells into a third partition, a fourth immune cell of the plurality of immune cells into a fourth partition, up to hundreds, thousands, tens of thousands, hundreds of thousands, or millions of immune cells that are each partitioned into a separate, individual, partition. It should be understood that each and every partitioned immune cell need be bound to one or more in particular of the target antigens. However, at least one immune cell of the population of immune cells partitioned into a partition will be bound to a target antigen.

### Systems

In an aspect, the disclosure provides for a system. The system may be useful to implement the methods provided herein, e.g., methods that characterize a binding characteristic of an ABM, e.g., antibody or antigen-binding fragment of an antibody. The system may include (a) a first pre-determined antigen binding molecule (PABM)-associated bead, wherein the first PABM-associated bead comprises a first PABM, and a first PABM reporter oligonucleotide; (b) a second pre-determined antigen binding molecule (PABM)-associated bead, wherein the second PABM-associated bead comprises a second PABM, and a second PABM reporter oligonucleotide; (c) a target antigen.

In any embodiment of the systems provided herein, the target antigen may be any antigen to which binding by an PABM is desirable. As discussed earlier herein, the target antigen that may be an antigen associated with an infectious agent, such as a viral, bacterial, parasitic, protozoal or prion agent. It may be an antigen associated with a tumor or a cancer, e.g., growth factor receptor, transcription factor. Further, it may be an antigen associated with a degenerative condition or disease (e.g., an amyloid protein or a tau protein), a cytokine, a co-stimulatory molecule, a co-inhibitory molecule, a growth factor, or an ion channel. It may be of any length, have any number of domains, and need not full-length, e.g., as expressed by a cell.

The systems provided herein may further include a plurality of nucleic acid barcode molecules. A nucleic acid barcode molecule of the plurality may include a partition-specific barcode sequence. In addition, to the partition-specific barcode sequence, it may include a capture sequence. Nucleic acid barcode molecules of the plurality of nucleic acid barcode molecules may further include functional sequences as disclosed herein. The system may further include reagents for generating a first of a plurality of barcoded nucleic acid molecules formed by complementary base pairing of: (a) the capture sequence of the plurality of nucleic acid barcode molecules and (b) an mRNA or DNA analyte comprising a sequence of an ABM, e.g., antibody or antigen-binding fragment of the antibody, for analysis by the system. The capture sequence, may be a polyT sequence, or it may be a polyG sequence. The capture sequence may be a sequence complementary to a sequence of an immunoglobulin variable or constant region or a B cell receptor variable or constant region.

In the systems provided here, the target antigen may further be coupled to a first reporter oligonucleotide. The first reporter oligonucleotide may include a first reporter barcode sequence. Such a reporter barcode sequence may identify the target antigen. The first reporter oligonucleotide may further include, e.g., further to the first reporter barcode sequence, a capture handle sequence. The capture handle sequence may couple to a capture sequence of a nucleic acid barcode molecule of the plurality of nucleic acid barcode molecules. The capture handle sequence of the first reporter oligonucleotide may couple to the capture sequence of the nucleic acid barcode molecule by complementary base pairing.

The systems provided herein may further include a partitioning system, which may be a microfluidic device. Microfluidic devices, channel structures and networks are discussed extensively herein at, for example, the section entitled “MICROFLUIDIC SYSTEMS.”

The systems provided herein may further include an analysis engine, a network and/or a sequencer.

### Kits

In an aspect, the disclosure provides for kits. The kits may be useful in the characterization of a binding characteristic of an ABM, e.g. antibody or antigen-binding fragment of an antibody.

The kits of the disclosure may include a plurality of pre-determined antigen binding molecule (PABM)-associated beads, each comprising a PABM barcode sequence.

The kits of the disclosure may also include a first antigen capable of binding to a first PABM with a known binding characteristic.

The antigens and PABMs may be coupled to a reporter oligonucleotide. The reporter oligonucleotide may include a reporter barcode sequence. The reporter barcode sequence may identify the antigen. The reporter oligonucleotide may further include a capture handle sequence, and, optionally, functional sequences (e.g, primer sequence or UMI).

The first antigen, as discussed earlier herein, may be an antigen associated with an infectious agent, such as a viral, e.g., SARS-COV2, bacterial, parasitic, protozoal or prion agent. It may be an antigen associated with a tumor or a cancer, e.g., growth factor receptor, transcription factor. Further, it may be an antigen associated with a degenerative condition or disease (e.g., an amyloid protein or a tau protein), a cytokine, a co-stimulatory molecule, a co-inhibitory molecule, a growth factor, or an ion channel. It may be of any length, have any number of domains, and need not full-length, e.g., as expressed by a cell.

It will further be understood that the compositions provided herein are not limited to including only one antigen, or only first and second antigens. The compositions provided herein may include one, two, at least two, three, at least three, four, at least four, five, at least five, six, at least six, seven, at least seven, eight, at least eight, nine, at least nine, ten, at least ten, twenty, at least twenty, thirty, at least thirty, forty, at least forty, fifty, at least fifty, sixty, at least sixty, seventy, at least seventy, eighty, at least eighty, ninety, at least ninety, one hundred, at least one hundred, five hundred, or at least five hundred, at least a thousand, at least tens of thousands, at least hundreds of thousands, or at least millions of antigens. The compositions provided herein may include at most ten, at most twenty, at most thirty, at most forty, at most fifty, at most sixty, at most seventy, at most eighty, at most ninety, at most one hundred, or most five hundred, at most one thousand, at most five thousand, at most ten thousand, at most one hundred thousand, or at most one million antigens. The antigens of the compositions may include any number of control antigens and/or any number of target antigens. Any of the compositions comprising any one or more antigens as provided herein may be included in a kit. If provided in a kit, the antigens may be in the kit with instructions for use thereof, e.g., to characterize an ABM.

Any of the kits provided herein, may further include a cell. The cell may be an immune cell. The cell may be a B cell, e.g., cell of B cell lineage such as a memory B cell, which expresses an antibody as a cell surface receptor. The cell may be a T cell. The composition, if it further include an immune cell, e.g., B or T cell, may include the immune cell bound to the antigen.

Any of the compositions provided herein may be in a partition. Partitions are discussed extensively herein, and include wells, microwell, and droplets.

Further disclosure related to these reagents can be found in the “Further Disclosure—Partitions, Partitioning, Reagents and Processing” section, immediately below.

### Further Disclosure—Partitions, Partitioning, Reagents and Processing

### Systems and Methods for Sample Compartmentalization

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions. The partition can be a droplet in an emulsion or a well. A partition may comprise one or more other partitions.

A partition may include one or more particles. A partition may include one or more types of particles. For example, a partition of the present disclosure may comprise one or more biological particles and/or macromolecular constituents thereof. A partition may comprise one or more beads. A partition may comprise one or more gel beads. A partition may comprise one or more cell beads. A partition may include a single gel bead, a single cell bead, or both a single cell bead and single gel bead. A partition may include one or more reagents. Alternatively, a partition may be unoccupied. For example, a partition may not comprise a bead.

Unique identifiers, such as barcodes, may be injected into the droplets previous to, subsequent to, or concurrently with droplet generation, such as via a bead, as described elsewhere herein.

The methods and systems of the present disclosure may comprise methods and systems for generating one or more partitions such as droplets. The droplets may comprise a plurality of droplets in an emulsion. In some examples, the droplets may comprise droplets in a colloid. In some cases, the emulsion may comprise a microemulsion or a nanoemulsion. In some examples, the droplets may be generated with aid of a microfluidic device and/or by subjecting a mixture of immiscible phases to agitation (e.g., in a container). In some cases, a combination of the mentioned methods may be used for droplet and/or emulsion formation.

The partitions described herein may comprise small volumes, for example, less than about 10 microliters (μL), 5 μL, 1 μL, 10 nanoliters (nL), 5 nL, 1 nL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet based partitions, the droplets may have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with beads, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

Droplets can be formed by creating an emulsion by mixing and/or agitating immiscible phases. Mixing or agitation may comprise various agitation techniques, such as vortexing, pipetting, tube flicking, or other agitation techniques. In some cases, mixing or agitation may be performed without using a microfluidic device. In some examples, the droplets may be formed by exposing a mixture to ultrasound or sonication. Systems and methods for droplet and/or emulsion generation by agitation are described in International Application No. PCT/US20/17785, which is entirely incorporated herein by reference for all purposes.

### Microfluidic Systems

Microfluidic devices or platforms comprising microfluidic channel networks (e.g., on a chip) can be utilized to generate partitions such as droplets and/or emulsions as described herein. Methods and systems for generating partitions such as droplets, methods of encapsulating biological particles in partitions, methods of increasing the throughput of droplet generation, and various geometries, architectures, and configurations of microfluidic devices and channels are described in U.S. Patent Publication Nos. 2019/0367997 and 2019/0064173, each of which is entirely incorporated herein by reference for all purposes.

In some examples, individual particles can be partitioned to discrete partitions by introducing a flowing stream of particles in an aqueous fluid into a flowing stream or reservoir of a non-aqueous fluid, such that droplets may be generated at the junction of the two streams/reservoir, such as at the junction of a microfluidic device provided elsewhere herein.

The methods of the present disclosure may comprise generating partitions and/or encapsulating particles, such as biological particles, in some cases, individual biological particles such as single cells. In some examples, reagents may be encapsulated and/or partitioned (e.g., co-partitioned with biological particles) in the partitions. Various mechanisms may be employed in the partitioning of individual particles. An example may comprise porous membranes through which aqueous mixtures of cells may be extruded into fluids (e.g., non-aqueous fluids).

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions may be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition in order to ensure that those partitions that are occupied are primarily singly occupied. In some cases, partitions among a plurality of partitions may contain at most one biological particle (e.g., bead, DNA, cell or cellular material). In some embodiments, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

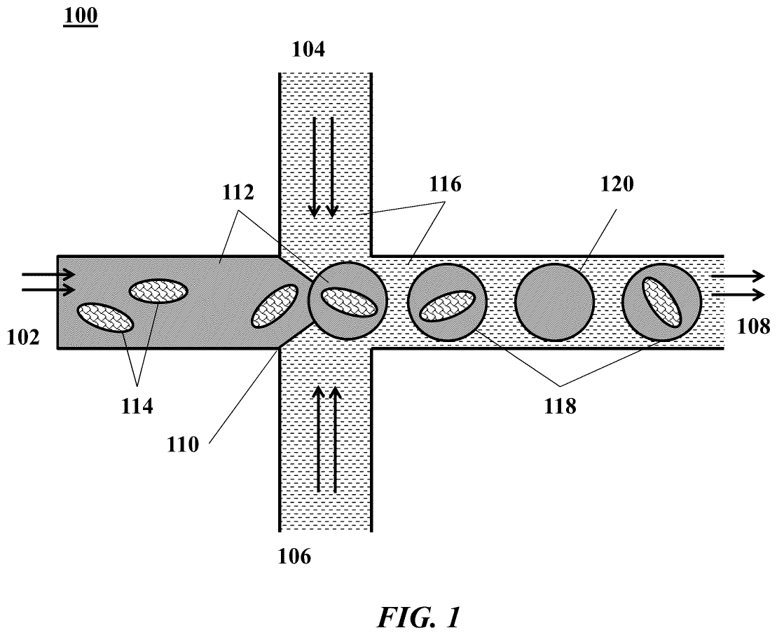

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (or cells) 114 may be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual biological particle 114 (such as droplets 118). A discrete droplet generated may include more than one individual biological particle 114 (not shown in FIG. 1). A discrete droplet may contain no biological particle 114 (such as droplet 120). Each discrete partition may maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 100 may have other geometrics. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 118, containing one or more biological particles 114, and (2) unoccupied droplets 120, not containing any biological particles 114. Occupied droplets 118 may comprise singly occupied droplets (having one biological particle) and multiply occupied droplets (having more than one biological particle). As described elsewhere herein, in some cases, the majority of occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it may be desirable to minimize the creation of excessive numbers of empty partitions, such as to reduce costs and/or increase efficiency. While this minimization may be achieved by providing a sufficient number of biological particles (e.g., biological particles 114) at the partitioning junction 110, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, flows can be controlled so as to present a non-Poissonian distribution of single-occupied partitions while providing lower levels of unoccupied partitions (e.g., no more than about 50%, about 25%, or about 10% unoccupied). The above noted ranges of unoccupied partitions can be achieved while still providing any of the single occupancy rates described above.

As will be appreciated, the above-described occupancy rates are also applicable to partitions that include both biological particles and additional reagents, such as beads (e.g., gel beads) carrying nucleic acid barcode molecules (e.g., oligonucleotides).

In some examples, a partition of the plurality of partitions may comprise a single biological particle (e.g., a single cell or a single nucleus of a cell). In some examples, a partition of the plurality of partitions may comprise multiple biological particles. Such partitions may be referred to as multiply occupied partitions, and may comprise, for example, two, three, four or more cells and/or beads (e.g., beads) comprising nucleic acid barcode molecules within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

Microfluidic systems for partitioning are further described in U.S. Patent Application Pub. No. US 2015/0376609, which is hereby incorporated by reference in its entirety.

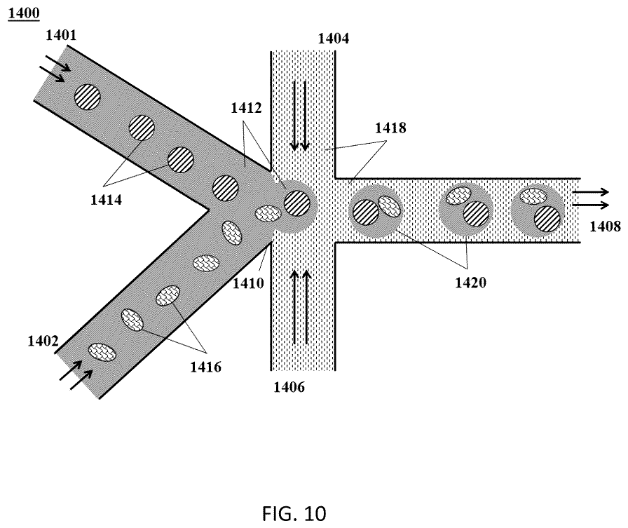

FIG. 10 shows an example of a microfluidic channel structure 1400 for delivering barcode carrying beads to droplets. The channel structure 1400 can include channel segments 1401, 1402, 1404, 1406 and 1408 communicating at a channel junction 1410. In operation, the channel segment 1401 may transport an aqueous fluid 1412 that includes a plurality of beads 1414 (e.g., with nucleic acid molecules, e.g., nucleic acid barcode molecules or barcoded oligonucleotides, molecular tags) along the channel segment 1401 into junction 1410. The plurality of beads 1414 may be sourced from a suspension of beads. For example, the channel segment 1401 may be connected to a reservoir comprising an aqueous suspension of beads 1414. The channel segment 1402 may transport the aqueous fluid 1412 that includes a plurality of biological particles 1416 along the channel segment 1402 into junction 1410. The plurality of biological particles 1416 may be sourced from a suspension of biological particles. For example, the channel segment 1402 may be connected to a reservoir comprising an aqueous suspension of biological particles 1416. In some instances, the aqueous fluid 1412 in either the first channel segment 1401 or the second channel segment 1402, or in both segments, can include one or more reagents, as further described below. A second fluid 1418 that is immiscible with the aqueous fluid 1412 (e.g., oil) can be delivered to the junction 1410 from each of channel segments 1404 and 1406. Upon meeting of the aqueous fluid 1412 from each of channel segments 1401 and 1402 and the second fluid 1418 from each of channel segments 1404 and 1406 at the channel junction 1410, the aqueous fluid 1412 can be partitioned as discrete droplets 1420 in the second fluid 1418 and flow away from the junction 1410 along channel segment 1408. The channel segment 1408 may deliver the discrete droplets to an outlet reservoir fluidly coupled to the channel segment 1408, where they may be harvested. As an alternative, the channel segments 1401 and 1402 may meet at another junction upstream of the junction 1410. At such junction, beads and biological particles may form a mixture that is directed along another channel to the junction 1410 to yield droplets 1420. The mixture may provide the beads and biological particles in an alternating fashion, such that, for example, a droplet comprises a single bead and a single biological particle.

### Controlled Partitioning

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

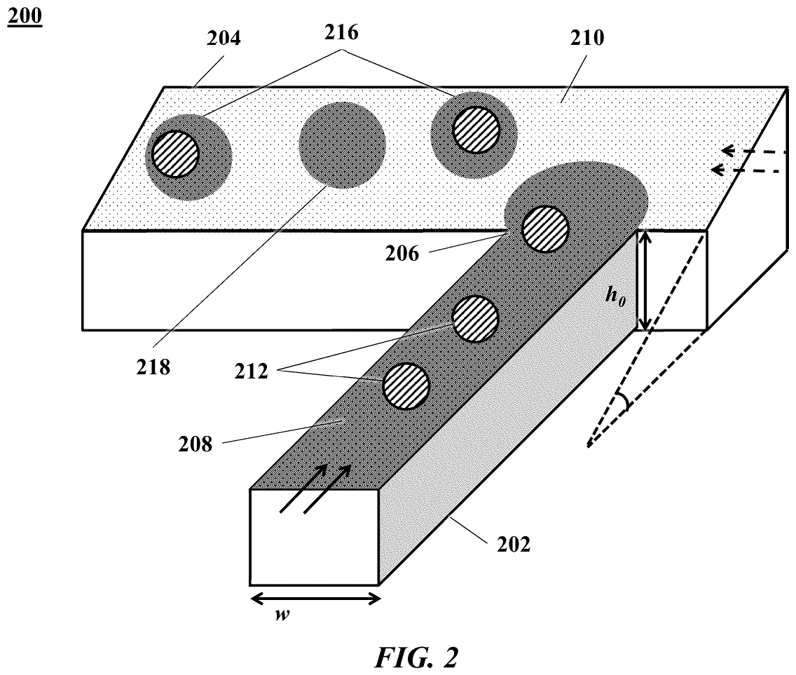

FIG. 2 shows an example of a microfluidic channel structure for the controlled partitioning of beads into discrete droplets. A channel structure 200 can include a channel segment 202 communicating at a channel junction 206 (or intersection) with a reservoir 204. The reservoir 204 can be a chamber. Any reference to “reservoir,” as used herein, can also refer to a “chamber.” In operation, an aqueous fluid 208 that includes suspended beads 212 may be transported along the channel segment 202 into the junction 206 to meet a second fluid 210 that is immiscible with the aqueous fluid 208 in the reservoir 204 to create droplets 216, 218 of the aqueous fluid 208 flowing into the reservoir 204. At the junction 206 where the aqueous fluid 208 and the second fluid 210 meet, droplets can form based on factors such as the hydrodynamic forces at the junction 206, flow rates of the two fluids 208, 210, fluid properties, and certain geometric parameters (e.g., w, h₀, α, etc.) of the channel structure 200. A plurality of droplets can be collected in the reservoir 204 by continuously injecting the aqueous fluid 208 from the channel segment 202 through the junction 206.

In some instances, the aqueous fluid 208 can have a substantially uniform concentration or frequency of beads 212. The beads 212 can be introduced into the channel segment 202 from a separate channel (not shown in FIG. 2). The frequency of beads 212 in the channel segment 202 may be controlled by controlling the frequency in which the beads 212 are introduced into the channel segment 202 and/or the relative flow rates of the fluids in the channel segment 202 and the separate channel. In some instances, the beads can be introduced into the channel segment 202 from a plurality of different channels, and the frequency controlled accordingly.

In some instances, the aqueous fluid 208 in the channel segment 202 can comprise biological particles. In some instances, the aqueous fluid 208 can have a substantially uniform concentration or frequency of biological particles. As with the beads, the biological particles can be introduced into the channel segment 202 from a separate channel. The frequency or concentration of the biological particles in the aqueous fluid 208 in the channel segment 202 may be controlled by controlling the frequency in which the biological particles are introduced into the channel segment 202 and/or the relative flow rates of the fluids in the channel segment 202 and the separate channel. In some instances, the biological particles can be introduced into the channel segment 202 from a plurality of different channels, and the frequency controlled accordingly. In some instances, a first separate channel can introduce beads and a second separate channel can introduce biological particles into the channel segment 202. The first separate channel introducing the beads may be upstream or downstream of the second separate channel introducing the biological particles.

The second fluid 210 can comprise an oil, such as a fluorinated oil, that includes a fluorosurfactant for stabilizing the resulting droplets, for example, inhibiting subsequent coalescence of the resulting droplets.

In some instances, the second fluid 210 may not be subjected to and/or directed to any flow in or out of the reservoir 204. For example, the second fluid 210 may be substantially stationary in the reservoir 204. In some instances, the second fluid 210 may be subjected to flow within the reservoir 204, but not in or out of the reservoir 204, such as via application of pressure to the reservoir 204 and/or as affected by the incoming flow of the aqueous fluid 208 at the junction 206. Alternatively, the second fluid 210 may be subjected and/or directed to flow in or out of the reservoir 204. For example, the reservoir 204 can be a channel directing the second fluid 210 from upstream to downstream, transporting the generated droplets.

Systems and methods for controlled partitioning are described further in PCT/US2018/047551, which is hereby incorporated by reference in its entirety.

### Cell Beads

In another aspect, in addition to or as an alternative to droplet-based partitioning, biological particles (e.g., cells) may be comprised within (e.g., encapsulated within) a particulate material to form a “cell bead”.

A cell bead can contain a biological particle (e.g., a cell) or macromolecular constituents (e.g., RNA, DNA, proteins, etc.) of a biological particle. A cell bead may include a single cell or multiple cells, or a derivative of the single cell or multiple cells. For example after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles and cell beads (and/or droplets or other partitions) containing macromolecular constituents of biological particles. Cell beads may be or include a cell, cell derivative, cellular material and/or material derived from the cell in, within, or encased in a matrix, such as a polymeric matrix. In some cases, a cell bead may comprise a live cell. In some instances, the live cell may be capable of being cultured when enclosed in a gel or polymer matrix, or of being cultured when comprising a gel or polymer matrix. In some instances, the polymer or gel may be diffusively permeable to certain components and diffusively impermeable to other components (e.g., macromolecular constituents).

Cell beads can provide certain potential advantages of being more storable and more portable than droplet-based partitioned biological particles. Furthermore, in some cases, it may be desirable to allow biological particles to incubate for a select period of time before analysis, such as in order to characterize changes in such biological particles over time, either in the presence or absence of different stimuli (or reagents).

Suitable polymers or gels may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel may comprise any other polymer or gel.

Encapsulation of biological particles may be performed by a variety of processes. Such processes may combine an aqueous fluid containing the biological particles with a polymeric precursor material that may be capable of being formed into a gel or other solid or semi-solid matrix upon application of a particular stimulus to the polymer precursor. The conditions sufficient to polymerize or gel the precursors may comprise any conditions sufficient to polymerize or gel the precursors. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), electromagnetic radiation, mechanical stimuli, or any combination thereof.

In some cases, air knife droplet or aerosol generators may be used to dispense droplets of precursor fluids into gelling solutions in order to form cell beads that include individual biological particles or small groups of biological particles. Likewise, membrane-based encapsulation systems may be used to generate cell beads comprising encapsulated biological particles as described herein. Microfluidic systems of the present disclosure, such as that shown in FIG. 1, may be readily used in encapsulating biological particles (e.g., cells) as described herein. Exemplary methods for encapsulating biological particles (e.g., cells) are also further described in U.S. Patent Application Pub. No. US 2015/0376609 and PCT/US2018/016019, which are hereby incorporated by reference in their entirety. In particular, and with reference to FIG. 1, the aqueous fluid 112 comprising (i) the biological particles 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the bead that includes the entrained biological particles. Examples of polymer precursor/initiator pairs include those described in U.S. Patent Application Publication No. 2014/0378345, which is entirely incorporated herein by reference for all purposes.

In some cases, encapsulated biological particles can be selectively releasable from the cell bead, such as through passage of time or upon application of a particular stimulus, that degrades the bead sufficiently to allow the biological particles (e.g., cell), or its other contents to be released from the bead, such as into a partition (e.g., droplet). Exemplary stimuli suitable for degradation of the bead are described in U.S. Patent Application Publication No. 2014/0378345, which is entirely incorporated herein by reference for all purposes.

The polymer or gel may be diffusively permeable to chemical or biochemical reagents. The polymer or gel may be diffusively impermeable to macromolecular constituents of the biological particle. In this manner, the polymer or gel may act to allow the biological particle to be subjected to chemical or biochemical operations while spatially confining the macromolecular constituents to a region of the droplet defined by the polymer or gel.

The polymer or gel may be functionalized to bind to targeted analytes, such as nucleic acids, proteins, carbohydrates, lipids or other analytes. The polymer or gel may be functionalized to bind to targeted analytes, such as nucleic acids, proteins, carbohydrates, lipids or other analytes. The polymer or gel, e.g., polymer gel matrix, hydrogel or hydrogel matrix, may be functionalized to couple or link to a plurality of capture agents. The plurality of capture agents may, e.g., covalently or non-covalently, couple or link to the backbone of the polymer. Sec, e.g., U.S. Pat. No. 10,590,244, which is incorporated by reference in its entirety, for exemplary cell bead functionalization strategies. In an embodiment, a first capture agent of a plurality of capture agents may be a polypeptide or aptamer that (i) couples or links to the backbone of the polymer, and (ii) binds a specific analyte (e.g., antibody or antigen-binding fragment thereof) secreted by the cell, e.g., B cell. By way of example, a first capture agent of a plurality of capture agents may be a polypeptide, e.g., antibody, or aptamer that couples/links to the backbone of the polymer and binds to a secreted antibody, e.g., at its Fc region. It will be understood that, in some embodiments, the first capture agent of the plurality of capture agents may, rather than couple/link to the backbone of the polymer of the gel matrix, embed in/couple to the cell membrane. In these embodiments, the first capture agent, e.g., polypeptide or aptamer, may (i) embed in the membrane of the cell and/or bind to a cell surface protein and (ii) bind the specific analyte, e.g., antibody or antigen-binding fragment thereof, thereby tethering the secreted analyte, e.g., antibody, to the cell.

The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The encapsulation of biological particles may constitute the partitioning of the biological particles into which other reagents are co-partitioned. Alternatively or in addition, encapsulated biological particles may be readily deposited into other partitions (e.g., droplets) as described above.

### Beads

Nucleic acid barcode molecules may be delivered to a partition (e.g., a droplet or well) via a solid support or carrier (e.g., a bead). In some cases, nucleic acid barcode molecules are initially associated with the solid support and then released from the solid support upon application of a stimulus, which allows the nucleic acid barcode molecules to dissociate or to be released from the solid support. In specific examples, nucleic acid barcode molecules are initially associated with the solid support (e.g., bead) and then released from the solid support upon application of a biological stimulus, a chemical stimulus, a thermal stimulus, an electrical stimulus, a magnetic stimulus, and/or a photo stimulus.

The solid support may be a bead. A solid support, e.g., a bead, may be porous, non-porous, hollow, solid, semi-solid, and/or a combination thereof. Beads may be solid, semi-solid, semi-fluidic, fluidic, and/or a combination thereof. In some instances, a solid support, e.g., a bead, may be at least partially dissolvable, disruptable, and/or degradable. In some cases, a solid support, e.g., a bead, may not be degradable. In some cases, the solid support, e.g., a bead, may be a gel bead. A gel bead may be a hydrogel bead. A gel bead may be formed from molecular precursors, such as a polymeric or monomeric species. A semi-solid support, e.g., a bead, may be a liposomal bead. Solid supports, e.g., beads, may comprise metals including iron oxide, gold, and silver. In some cases, the solid support, e.g., the bead, may be a silica bead. In some cases, the solid support, e.g., a bead, can be rigid. In other cases, the solid support, e.g., a bead, may be flexible and/or compressible.

A partition may comprise one or more unique identifiers, such as barcodes. Barcodes may be previously, subsequently or concurrently delivered to the partitions that hold the compartmentalized or partitioned biological particle. For example, barcodes may be injected into droplets or deposited in microwells previous to, subsequent to, or concurrently with droplet generation or providing of reagents in the microwells, respectively. The delivery of the barcodes to a particular partition allows for the later attribution of the characteristics of the individual biological particle to the particular partition. Barcodes may be delivered, for example on a nucleic acid molecule (e.g., via a nucleic acid barcode molecule), to a partition via any suitable mechanism. Nucleic acid barcode molecules can be delivered to a partition via a bead. Beads are described in further detail below.

In some cases, nucleic acid barcode molecules can be initially associated with the bead and then released from the bead. Release of the nucleic acid barcode molecules can be passive (e.g., by diffusion out of the bead). In addition or alternatively, release from the bead can be upon application of a stimulus which allows the nucleic acid barcode molecules to dissociate or to be released from the bead. Such stimulus may disrupt the bead, an interaction that couples the nucleic acid barcode molecules to or within the bead, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

Methods and systems for partitioning barcode carrying beads into droplets are provided herein, and in in US. Patent Publication Nos. 2019/0367997 and 2019/0064173, and International Application No. PCT/US20/17785, each of which is herein entirely incorporated by reference for all purposes.

A bead may be porous, non-porous, solid, semi-solid, semi-fluidic, fluidic, and/or a combination thereof. In some instances, a bead may be dissolvable, disruptable, and/or degradable. Degradable beads, as well as methods for degrading beads, are described in PCT/US2014/044398, which is hereby incorporated by reference in its entirety. In some cases, any combination of stimuli, e.g., stimuli described in PCT/US2014/044398 and US Patent Application Pub. No. 2015/0376609, hereby incorporated by reference in its entirety, may trigger degradation of a bead. For example, a change in pH may enable a chemical agent (e.g., DTT) to become an effective reducing agent.

In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. Sec, e.g., PCT/US2014/044398, which is hereby incorporated by reference in its entirety. Beads may also be formed from materials other than polymers, including lipids, micelles, ceramics, glass-ceramics, material composites, metals, other inorganic materials, and others.

In some cases, the bead may comprise covalent or ionic bonds between polymeric precursors (e.g., monomers, oligomers, linear polymers), nucleic acid barcode molecules (e.g., oligonucleotides), primers, and other entities. In some cases, the covalent bonds can be carbon-carbon bonds, thioether bonds, or carbon-heteroatom bonds.

In some cases, a plurality of nucleic acid barcode molecules may be attached to a bead. The nucleic acid barcode molecules may be attached directly or indirectly to the bead. In some cases, the nucleic acid barcode molecules may be covalently linked to the bead. In some cases, the nucleic acid barcode molecules are covalently linked to the bead via a linker. In some cases, the linker is a degradable linker. In some cases, the linker comprises a labile bond configured to release said nucleic acid barcode molecule of said plurality of nucleic acid barcode molecules. In some cases, the labile bond comprises a disulfide linkage.

Activation of disulfide linkages within a bead can be controlled such that only a small number of disulfide linkages are activated. Methods of controlling activation of disulfide linkages within a bead are described in PCT/US2014/044398, which is hereby incorporated by reference in its entirety.

In some cases, a bead may comprise an acrydite moiety, which in certain aspects may be used to attach one or more nucleic acid barcode molecules (e.g., barcode sequence, nucleic acid barcode molecule, barcoded oligonucleotide, primer, or other oligonucleotide) to the bead. Acrydite moieties, as well as their uses in attaching nucleic acid molecules to beads, are described in PCT/US2014/044398, which is hereby incorporated by reference in its entirety.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule, e.g., a nucleic acid barcode molecule described herein.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. Exemplary precursors comprising functional groups are described in PCT/US2014/044398, which is hereby incorporated by reference in its entirety.

Other non-limiting examples of labile bonds that may be coupled to a precursor or bead are described in PCT/US2014/044398, which is hereby incorporated by reference in its entirety. A bond may be cleavable via other nucleic acid molecule targeting enzymes, such as restriction enzymes (e.g., restriction endonucleases), as described further below.

Species may be encapsulated in beads during bead generation (e.g., during polymerization of precursors). Such species may or may not participate in polymerization. Sec, e.g., PCT/US2014/044398, which is hereby incorporated by reference in its entirety. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Alternatively or in addition, species may be partitioned in a partition (e.g., droplet) during or subsequent to partition formation. Such species may include, without limitation, the abovementioned species that may also be encapsulated in a bead.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

### Nucleic Acid Barcode Molecules

A nucleic acid barcode molecule may contain one or more barcode sequences. A plurality of nucleic acid barcode molecules may be coupled to a bead. The one or more barcode sequences may include sequences that are the same for all nucleic acid molecules coupled to a given bead and/or sequences that are different across all nucleic acid molecules coupled to the given bead. The nucleic acid molecule may be incorporated into the bead.

Nucleic acid barcode molecules can comprise one or more functional sequences for coupling to an analyte or analyte tag such as a reporter oligonucleotide. Such functional sequences can include, e.g., a template switch oligonucleotide (TSO) sequence, a primer sequence (e.g., a poly T sequence, or a nucleic acid primer sequence complementary to a target nucleic acid sequence and/or for amplifying a target nucleic acid sequence, a random primer, and a primer sequence for messenger RNA).

In some cases, the nucleic acid barcode molecule can further comprise a unique molecular identifier (UMI). In some cases, the nucleic acid barcode molecule can comprise one or more functional sequences, for example, for attachment to a sequencing flow cell, such as, for example, a P5 sequence (or a portion thereof) for Illumina® sequencing. In some cases, the nucleic acid barcode molecule or derivative thereof (e.g., oligonucleotide or polynucleotide generated from the nucleic acid molecule) can comprise another functional sequence, such as, for example, a P7 sequence (or a portion thereof) for attachment to a sequencing flow cell for Illumina sequencing. In some cases, the nucleic acid molecule can comprise an R1 primer sequence for Illumina sequencing. In some cases, the nucleic acid molecule can comprise an R2 primer sequence for Illumina sequencing. In some cases, a functional sequence can comprise a partial sequence, such as a partial barcode sequence, partial anchoring sequence, partial sequencing primer sequence (e.g., partial R1 sequence, partial R2 sequence, etc.), a partial sequence configured to attach to the flow cell of a sequencer (e.g., partial P5 sequence, partial P7 sequence, etc.), or a partial sequence of any other type of sequence described elsewhere herein. A partial sequence may contain a contiguous or continuous portion or segment, but not all, of a full sequence, for example. In some cases, a downstream procedure may extend the partial sequence, or derivative thereof, to achieve a full sequence of the partial sequence, or derivative thereof.

Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

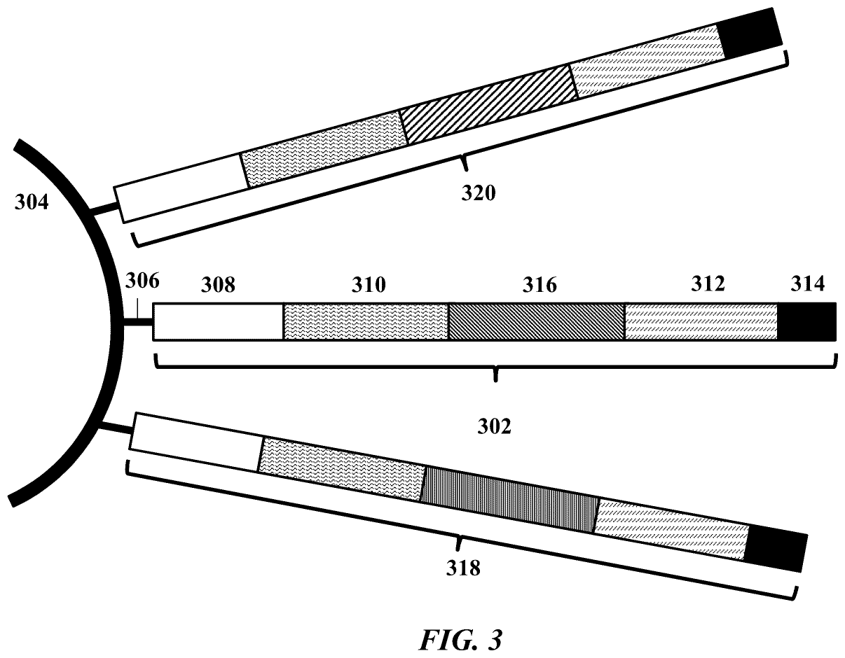

FIG. 3 illustrates an example of a barcode carrying bead. A nucleic acid barcode molecule 302 can be coupled to a bead 304 by a releasable linkage 306, such as, for example, a disulfide linker. The same bead 304 may be coupled (e.g., via releasable linkage) to one or more other nucleic acid barcode molecules 318, 320. The nucleic acid barcode molecule 302 may be or comprise a barcode. As noted elsewhere herein, the structure of the barcode may comprise a number of sequence elements. The nucleic acid barcode molecule 302 may comprise a functional sequence 308 that may be used in subsequent processing. For example, the functional sequence 308 may include one or more of a sequencer specific flow cell attachment sequence (e.g., a P5 sequence for Illumina® sequencing systems) and a sequencing primer sequence (e.g., a R1 primer for Illumina® sequencing systems), or partial sequence(s) thereof. The nucleic acid barcode molecule 302 may comprise a barcode sequence 310 for use in barcoding the sample (e.g., DNA, RNA, protein, etc.). In some cases, the barcode sequence 310 can be bead-specific such that the barcode sequence 310 is common to all nucleic acid barcode molecules (e.g., including nucleic acid barcode molecule 302) coupled to the same bead 304. Alternatively or in addition, the barcode sequence 310 can be partition-specific such that the barcode sequence 310 is common to all nucleic acid barcode molecules coupled to one or more beads that are partitioned into the same partition. The nucleic acid barcode molecule 302 may comprise sequence 312 complementary to an analyte of interest, e.g., a priming sequence. Sequence 312 can be a poly-T sequence complementary to a poly-A tail of an mRNA analyte, a targeted priming sequence, and/or a random priming sequence. The nucleic acid barcode molecule 302 may comprise an anchoring sequence 314 to ensure that the specific priming sequence 312 hybridizes at the sequence end (e.g., of the mRNA). For example, the anchoring sequence 314 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid barcode molecule 302 may comprise a unique molecular identifying sequence 316 (e.g., unique molecular identifier (UMI)). In some cases, the unique molecular identifying sequence 316 may comprise from about 5 to about 8 nucleotides. Alternatively, the unique molecular identifying sequence 316 may compress less than about 5 or more than about 8 nucleotides. The unique molecular identifying sequence 316 may be a unique sequence that varies across individual nucleic acid barcode molecules (e.g., 302, 318, 320, etc.) coupled to a single bead (e.g., bead 304). In some cases, the unique molecular identifying sequence 316 may be a random sequence (e.g., such as a random N-mer sequence). For example, the UMI may provide a unique identifier of the starting analyte (e.g., mRNA) molecule that was captured, in order to allow quantitation of the number of original expressed RNA molecules. As will be appreciated, although FIG. 3 shows three nucleic acid barcode molecules 302, 318, 320 coupled to the surface of the bead 304, an individual bead may be coupled to any number of individual nucleic acid barcode molecules, for example, from one to tens to hundreds of thousands, millions, or even a billion of individual nucleic acid barcode molecules. The respective barcodes for the individual nucleic acid barcode molecules can comprise both common sequence segments or relatively common sequence segments (e.g., 308, 310, 312, etc.) and variable or unique sequence segments (e.g., 316) between different individual nucleic acid barcode molecules coupled to the same bead.

In operation, a biological particle (e.g., cell, DNA, RNA, etc.) can be co-partitioned along with a barcode bearing bead 304. The nucleic acid barcode molecules 302, 318, 320 can be released from the bead 304 in the partition. By way of example, in the context of analyzing sample RNA, the poly-T segment (e.g., 312) of one of the released nucleic acid barcode molecules (e.g., 302) can hybridize to the poly-A tail of a mRNA molecule. Reverse transcription may result in a cDNA transcript of the mRNA, but which transcript includes each of the sequence segments 308, 310, 316 of the nucleic acid barcode molecule 302. Because the nucleic acid barcode molecule 302 comprises an anchoring sequence 314, it will more likely hybridize to and prime reverse transcription at the sequence end of the poly-A tail of the mRNA. cDNA transcripts of the individual mRNA molecules from any given partition may include a common barcode sequence segment 310. However, the transcripts made from the different mRNA molecules within a given partition may vary at the unique molecular identifying sequence 312 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid barcode molecules bound to the bead (e.g., gel bead) may be used to hybridize and capture the mRNA on the solid phase of the bead, for example, in order to facilitate the separation of the RNA from other cell contents. In such cases, further processing may be performed, in the partitions or outside the partitions (e.g., in bulk). For instance, the RNA molecules on the beads may be subjected to reverse transcription or other nucleic acid processing, additional adapter sequences may be added to the barcoded nucleic acid molecules, or other nucleic acid reactions (e.g., amplification, nucleic acid extension) may be performed. The beads or products thereof (e.g., barcoded nucleic acid molecules) may be collected from the partitions, and/or pooled together and subsequently subjected to clean up and further characterization (e.g., sequencing).

The operations described herein may be performed at any useful or convenient step. For instance, the beads comprising nucleic acid barcode molecules may be introduced into a partition (e.g., well or droplet) prior to, during, or following introduction of a sample into the partition. The nucleic acid molecules of a sample may be subjected to barcoding, which may occur on the bead (in cases where the nucleic acid molecules remain coupled to the bead) or following release of the nucleic acid barcode molecules into the partition. In cases where analytes from the sample are captured by the nucleic acid barcode molecules in a partition (e.g., by hybridization), captured analytes from various partitions may be collected, pooled, and subjected to further processing (e.g., reverse transcription, adapter attachment, amplification, clean up, sequencing). For example, in cases wherein the nucleic acid molecules from the sample remain attached to the bead, the beads from various partitions may be collected, pooled, and subjected to further processing (e.g., reverse transcription, adapter attachment, amplification, clean up, sequencing). In other instances, one or more of the processing methods, e.g., reverse transcription, may occur in the partition. For example, conditions sufficient for barcoding, adapter attachment, reverse transcription, or other nucleic acid processing operations may be provided in the partition and performed prior to clean up and sequencing.

In some instances, a bead may comprise a capture sequence or binding sequence configured to bind to a corresponding capture sequence or binding sequence. In some instances, a bead may comprise a plurality of different capture sequences or binding sequences configured to bind to different respective corresponding capture sequences or binding sequences. For example, a bead may comprise a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead may comprise any number of different capture sequences. In some instances, a bead may comprise at least 2, 3, 4, 5, 6, 7, 8, 9, 10 or more different capture sequences or binding sequences configured to bind to different respective capture sequences or binding sequences, respectively. Alternatively or in addition, a bead may comprise at most about 10, 9, 8, 7, 6, 5, 4, 3, or 2 different capture sequences or binding sequences configured to bind to different respective capture sequences or binding sequences. In some instances, the different capture sequences or binding sequences may be configured to facilitate analysis of a same type of analyte. In some instances, the different capture sequences or binding sequences may be configured to facilitate analysis of different types of analytes (with the same bead). The capture sequence may be designed to attach to a corresponding capture sequence. Beneficially, such corresponding capture sequence may be introduced to, or otherwise induced in, a biological particle (e.g., cell, cell bead, etc.) for performing different assays in various formats (e.g., barcoded antibodies comprising the corresponding capture sequence, barcoded MHC dextramers comprising the corresponding capture sequence, barcoded guide RNA molecules comprising the corresponding capture sequence, etc.), such that the corresponding capture sequence may later interact with the capture sequence associated with the bead. In some instances, a capture sequence coupled to a bead (or other support) may be configured to attach to a linker molecule, such as a splint molecule, wherein the linker molecule is configured to couple the bead (or other support) to other molecules through the linker molecule, such as to one or more analytes or one or more other linker molecules.

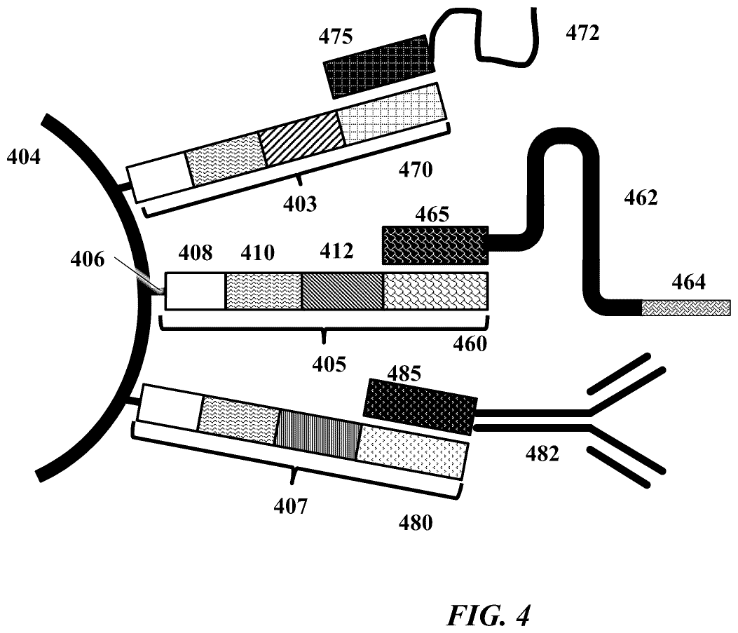

FIG. 4 illustrates another example of a barcode carrying bead. A nucleic acid barcode molecule 405, such as an oligonucleotide, can be coupled to a bead 404 by a releasable linkage 406, such as, for example, a disulfide linker. The nucleic acid barcode molecule 405 may comprise a first capture sequence 460. The same bead 404 may be coupled (e.g., via releasable linkage) to one or more other nucleic acid molecules 403, 407 comprising other capture sequences. The nucleic acid barcode molecule 405 may be or comprise a barcode. As noted elsewhere herein, the structure of the barcode may comprise a number of sequence elements, such as a functional sequence 408 (e.g., flow cell attachment sequence, sequencing primer sequence, etc.), a barcode sequence 410 (e.g., bead-specific sequence common to bead, partition-specific sequence common to partition, etc.), and a unique molecular identifier 412 (e.g., unique sequence within different molecules attached to the bead), or partial sequences thereof. The capture sequence 460 may be configured to attach to a corresponding capture sequence 465. In some instances, the corresponding capture sequence 465 may be coupled to another molecule that may be an analyte or an intermediary carrier. For example, as illustrated in FIG. 4, the corresponding capture sequence 465 is coupled to a guide RNA molecule 462 comprising a target sequence 464, wherein the target sequence 464 is configured to attach to the analyte. Another oligonucleotide molecule 407 attached to the bead 404 comprises a second capture sequence 480 which is configured to attach to a second corresponding capture sequence 485. As illustrated in FIG. 4, the second corresponding capture sequence 485 is coupled to an antibody 482. In some cases, the antibody 482 may have binding specificity to an analyte (e.g., surface protein). Alternatively, the antibody 482 may not have binding specificity. Another oligonucleotide molecule 403 attached to the bead 404 comprises a third capture sequence 470 which is configured to attach to a third corresponding capture sequence 475. As illustrated in FIG. 4, the third corresponding capture sequence 475 is coupled to a molecule 472. The molecule 472 may or may not be configured to target an analyte. The other oligonucleotide molecules 403, 407 may comprise the other sequences (e.g., functional sequence, barcode sequence, UMI, etc.) described with respect to oligonucleotide molecule 405. While a single oligonucleotide molecule comprising each capture sequence is illustrated in FIG. 4, it will be appreciated that, for each capture sequence, the bead may comprise a set of one or more oligonucleotide molecules each comprising the capture sequence. For example, the bead may comprise any number of sets of one or more different capture sequences. Alternatively or in addition, the bead 404 may comprise other capture sequences. Alternatively or in addition, the bead 404 may comprise fewer types of capture sequences (e.g., two capture sequences). Alternatively or in addition, the bead 404 may comprise oligonucleotide molecule(s) comprising a priming sequence, such as a specific priming sequence such as an mRNA specific priming sequence (e.g., poly-T sequence), a targeted priming sequence, and/or a random priming sequence, for example, to facilitate an assay for gene expression.

In operation, the barcoded oligonucleotides may be released (e.g., in a partition), as described elsewhere herein. Alternatively, the nucleic acid molecules bound to the bead (e.g., gel bead) may be used to hybridize and capture analytes (e.g., one or more types of analytes) on the solid phase of the bead.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. Sec, e.g., PCT/US2014/044398, which is hereby incorporated by reference in its entirety.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. See, e.g., PCT/US2014/044398, which is hereby incorporated by reference in its entirety.

As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

In some cases, a species (e.g., oligonucleotide molecules comprising barcodes) that are attached to a solid support (e.g., a bead) may comprise a U-excising element that allows the species to release from the bead. In some cases, the U-excising element may comprise a single-stranded DNA (ssDNA) sequence that contains at least one uracil. The species may be attached to a solid support via the ssDNA sequence containing the at least one uracil. The species may be released by a combination of uracil-DNA glycosylase (e.g., to remove the uracil) and an endonuclease (e.g., to induce an ssDNA break). If the endonuclease generates a 5′ phosphate group from the cleavage, then additional enzyme treatment may be included in downstream processing to eliminate the phosphate group, e.g., prior to ligation of additional sequencing handle elements, e.g., Illumina full P5 sequence, partial P5 sequence, full R1 sequence, and/or partial R1 sequence.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying nucleic acids (e.g., mRNA, the genomic DNA) from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides (e.g., attached to a bead) into partitions, e.g., droplets within microfluidic systems.

In an example, beads are provided that each include large numbers of the above described nucleic acid barcode molecules releasably attached to the beads, where all of the nucleic acid barcode molecules attached to a particular bead will include a common nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid barcode molecules into the partitions, as they are capable of carrying large numbers of nucleic acid barcode molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. In some cases, the population of beads provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences.

Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. In some embodiments, the number of nucleic acid molecules including the barcode sequence on an individual bead is between about 1,000 to about 10,000 nucleic acid molecules, about 5,000 to about 50,000 nucleic acid molecules, about 10,000 to about 100,000 nucleic acid molecules, about 50,000 to about 1,000,000 nucleic acid molecules, about 100,000 to about 10,000,000 nucleic acid molecules, about 1,000,000 to about 1 billion nucleic acid molecules.

Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set. In some embodiments, such different barcode sequences can be associated with a given bead.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid barcode molecules, at least about 5,000 nucleic acid barcode molecules, at least about 10,000 nucleic acid barcode molecules, at least about 50,000 nucleic acid barcode molecules, at least about 100,000 nucleic acid barcode molecules, at least about 500,000 nucleic acids, at least about 1,000,000 nucleic acid barcode molecules, at least about 5,000,000 nucleic acid barcode molecules, at least about 10,000,000 nucleic acid barcode molecules, at least about 50,000,000 nucleic acid barcode molecules, at least about 100,000,000 nucleic acid barcode molecules, at least about 250,000,000 nucleic acid barcode molecules and in some cases at least about 1 billion nucleic acid barcode molecules.

In some cases, the resulting population of partitions provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences. Additionally, each partition of the population can include between about 1,000 to about 10,000 nucleic acid barcode molecules, about 5,000 to about 50,000 nucleic acid barcode molecules, about 10,000 to about 100,000 nucleic acid barcode molecules, about 50,000 to about 1,000,000 nucleic acid barcode molecules, about 100,000 to about 10,000,000 nucleic acid barcode molecules, about 1,000,000 to about 1 billion nucleic acid barcode molecules.

In some cases, it may be desirable to incorporate multiple different barcodes within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) may be releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and may be degraded for release of the attached nucleic acid molecules through exposure to a reducing agent, such as DTT.

### Reagents

In accordance with certain aspects, biological particles may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles may be partitioned along with other reagents, as will be described further below.

The methods and systems of the present disclosure may comprise microfluidic devices and methods of use thereof, which may be used for co-partitioning biological particles with reagents. Such systems and methods are described in U.S. Patent Publication No. US/20190367997, which is herein incorporated by reference in its entirety for all purposes.

Beneficially, when lysis reagents and biological particles are co-partitioned, the lysis reagents can facilitate the release of the contents of the biological particles within the partition. The contents released in a partition may remain discrete from the contents of other partitions.

As will be appreciated, the channel segments of the microfluidic devices described elsewhere herein may be coupled to any of a variety of different fluid sources or receiving components, including reservoirs, tubing, manifolds, or fluidic components of other systems. As will be appreciated, the microfluidic channel structures may have various geometries and/or configurations. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the biological particles to cause the release of the biological particle's contents into the partitions. For example, in some cases, surfactant-based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion-based partitioning such as encapsulation of biological particles that may be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., a cell or a nucleus in a polymer matrix), the biological particles may be exposed to an appropriate stimulus to release the biological particles or their contents from a co-partitioned bead. For example, in some cases, a chemical stimulus may be co-partitioned along with an encapsulated biological particle to allow for the degradation of the bead and release of the cell or its contents into the larger partition. In some cases, this stimulus may be the same as the stimulus described elsewhere herein for release of nucleic acid molecules (e.g., oligonucleotides) from their respective bead. In alternative examples, this may be a different and non-overlapping stimulus, in order to allow an encapsulated biological particle to be released into a partition at a different time from the release of nucleic acid molecules into the same partition. For a description of methods, compositions, and systems for encapsulating cells (also referred to as a “cell bead”), see, e.g., U.S. Pat. No. 10,428,326 and U.S. Pat. Pub. 20190100632, which are each incorporated by reference in their entirety.

Additional reagents may also be co-partitioned with the biological particle, such as endonucleases to fragment a biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNAse, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching.

In some cases, template switching can be used to increase the length of a cDNA. In some cases, template switching can be used to append a predefined nucleic acid sequence to the cDNA. Template switching is further described in PCT/US2017/068320, which is hereby incorporated by reference in its entirety. Template switching oligonucleotides may comprise a hybridization region and a template region. Template switching oligonucleotides are further described in PCT/US2017/068320, which is hereby incorporated by reference in its entirety.

Any of the reagents described in this disclosure may be encapsulated in, or otherwise coupled to, a droplet, or bead, with any chemicals, particles, and elements suitable for sample processing reactions involving biomolecules, such as, but not limited to, nucleic acid molecules and proteins. For example, a bead or droplet used in a sample preparation reaction for DNA sequencing may comprise one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to: buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, and oligonucleotides.

Once the contents of the cells are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles. In some aspects, this is performed by co-partitioning the individual biological particle or groups of biological particles with the unique identifiers, such as described above (with reference to FIG. 1 or 2).

In some cases, additional beads can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction. In such cases, the flow and frequency of the different beads into the channel or junction may be controlled to provide for a certain ratio of beads from each source, while ensuring a given pairing or combination of such beads into a partition with a given number of biological particles (e.g., one biological particle and one bead per partition).

In some embodiments, following the generation of barcoded nucleic acid molecules according to methods disclosed herein, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken and the contents of the droplet pooled for additional operations.

### Wells

As described herein, one or more processes may be performed in a partition, which may be a well. The well may be a well of a plurality of wells of a substrate, such as a microwell of a microwell array or plate, or the well may be a microwell or microchamber of a device (e.g., microfluidic device) comprising a substrate. The well may be a well of a well array or plate, or the well may be a well or chamber of a device (e.g., fluidic device). In some embodiments, a well of a fluidic device is fluidically connected to another well of the fluidic device. Accordingly, the wells or microwells may assume an “open” configuration, in which the wells or microwells are exposed to the environment (e.g., contain an open surface) and are accessible on one planar face of the substrate, or the wells or microwells may assume a “closed” or “sealed” configuration, in which the microwells are not accessible on a planar face of the substrate. In some instances, the wells or microwells may be configured to toggle between “open” and “closed” configurations. For instance, an “open” microwell or set of microwells may be “closed” or “sealed” using a membrane (e.g., semi-permeable membrane), an oil (e.g., fluorinated oil to cover an aqueous solution), or a lid, as described elsewhere herein.

The well may have a volume of less than 1 milliliter (mL). For instance, the well may be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well may be configured to hold a volume of about 1000 μL, about 100 μL, about 10 μL, about 1 μL, about 100 nL, about 10 nL, about 1 nL, about 100 pL, about 10 pL, etc. The well may be configured to hold a volume of at least 10 pL, at least 100 pL, at least 1 nL, at least 10 nL, at least 100 nL, at least 1 μL, at least 10 μL, at least 100 μL, at least 1000 μL, or more. The well may be configured to hold a volume in a range of volumes listed herein, for example, from about 5 nL to about 20 nL, from about 1 nL to about 100 nL, from about 500 pL to about 100 μL, etc. The well may be of a plurality of wells that have varying volumes and may be configured to hold a volume appropriate to accommodate any of the partition volumes described herein.

In some instances, a microwell array or plate comprises a single variety of microwells. In some instances, a microwell array or plate comprises a variety of microwells. For instance, the microwell array or plate may comprise one or more types of microwells within a single microwell array or plate. The types of microwells may have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate may comprise any number of different types of microwells. For example, the microwell array or plate may comprise 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well may have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate comprises different types of microwells that are located adjacent to one another within the array or plate. For instance, a microwell with one set of dimensions may be located adjacent to and in contact with another microwell with a different set of dimensions. Similarly, microwells of different geometries may be placed adjacent to or in contact with one another. The adjacent microwells may be configured to hold different articles; for example, one microwell may be used to contain a cell, cell bead, or other sample (e.g., cellular components, nucleic acid molecules, etc.) while the adjacent microwell may be used to contain a droplet, bead, or other reagent. In some cases, the adjacent microwells may be configured to merge the contents held within, e.g., upon application of a stimulus, or spontaneously, upon contact of the articles in each microwell.

As is described elsewhere herein, a plurality of partitions may be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells may comprise both unoccupied wells (e.g., empty wells) and occupied wells.

A well may comprise any of the reagents described herein, or combinations thereof. These reagents may include, for example, barcode molecules, enzymes, adapters, and combinations thereof. The reagents may be physically separated from a sample (e.g., a cell, cell bead, or cellular components, e.g., proteins, nucleic acid molecules, etc.) that is placed in the well. This physical separation may be accomplished by containing the reagents within, or coupling to, a bead that is placed within a well. The physical separation may also be accomplished by dispensing the reagents in the well and overlaying the reagents with a layer that is, for example, dissolvable, meltable, or permeable prior to introducing the polynucleotide sample into the well. This layer may be, for example, an oil, wax, membrane (e.g., semi-permeable membrane), or the like. The well may be sealed at any point, for example, after addition of the bead, after addition of the reagents, or after addition of either of these components. The sealing of the well may be useful for a variety of purposes, including preventing escape of beads or loaded reagents from the well, permitting select delivery of certain reagents (e.g., via the use of a semi-permeable membrane), for storage of the well prior to or following further processing, etc.

Once sealed, the well may be subjected to conditions for further processing of a cell (or cells) in the well. For instance, reagents in the well may allow further processing of the cell, e.g., cell lysis, as further described herein. Alternatively, the well (or wells such as those of a well-based array) comprising the cell (or cells) may be subjected to freeze-thaw cycling to process the cell (or cells), e.g., cell lysis. The well containing the cell may be subjected to freezing temperatures (e.g., 0° C., below 0° C., −5° C., −10° C., −15° C., −20° C., −25° C., −30° C., −35° C., −40° C., −45° C., −50° C., −55° C., −60° C., −65° C., −70° C., −80° C., or −85° C.). Freezing may be performed in a suitable manner, e.g., sub-zero freezer or a dry ice/ethanol bath. Following an initial freezing, the well (or wells) comprising the cell (or cells) may be subjected to freeze-thaw cycles to lyse the cell (or cells). In one embodiment, the initially frozen well (or wells) are thawed to a temperature above freezing (e.g., 4° C. or above, 8° C. or above, 12° C. or above, 16° C. or above, 20° C. or above, room temperature, or 25° C. or above). In another embodiment, the freezing is performed for less than 10 minutes (e.g., 5 minutes or 7 minutes) followed by thawing at room temperature for less than 10 minutes (e.g., 5 minutes or 7 minutes). This freeze-thaw cycle may be repeated a number of times, e.g., 2, 3, 4 or more times, to obtain lysis of the cell (or cells) in the well (or wells). In one embodiment, the freezing, thawing and/or freeze/thaw cycling is performed in the absence of a lysis buffer. Additional disclosure related to freeze-thaw cycling is provided in WO2019165181A1, which is incorporated herein by reference in its entirety.

A well may comprise free reagents and/or reagents encapsulated in, or otherwise coupled to or associated with, beads, or droplets.

The wells may be provided as a part of a kit. For example, a kit may comprise instructions for use, a microwell array or device, and reagents (e.g., beads). The kit may comprise any useful reagents for performing the processes described herein, e.g., nucleic acid reactions, barcoding of nucleic acid molecules, sample processing (e.g., for cell lysis, fixation, and/or permeabilization).

In some cases, a well comprises a bead, or droplet that comprises a set of reagents that has a similar attribute (e.g., a set of enzymes, a set of minerals, a set of oligonucleotides, a mixture of different barcode molecules, a mixture of identical barcode molecules). In other cases, a bead or droplet comprises a heterogeneous mixture of reagents. In some cases, the heterogeneous mixture of reagents can comprise all components necessary to perform a reaction. In some cases, such mixture can comprise all components necessary to perform a reaction, except for 1, 2, 3, 4, 5, or more components necessary to perform a reaction. In some cases, such additional components are contained within, or otherwise coupled to, a different droplet or bead, or within a solution within a partition (e.g., microwell) of the system.

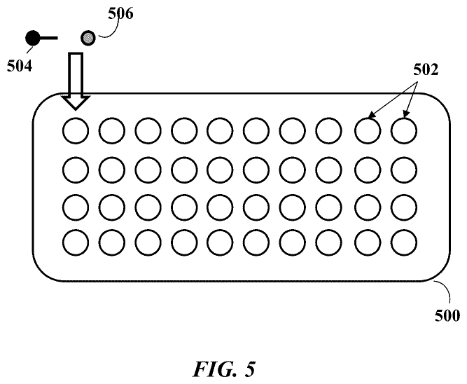

FIG. 5 schematically illustrates an example of a microwell array. The array can be contained within a substrate 500. The substrate 500 comprises a plurality of wells 502. The wells 502 may be of any size or shape, and the spacing between the wells, the number of wells per substrate, as well as the density of the wells on the substrate 500 can be modified, depending on the particular application. In one such example application, a sample molecule 506, which may comprise a cell or cellular components (e.g., nucleic acid molecules) is co-partitioned with a bead 504, which may comprise a nucleic acid barcode molecule coupled thereto. The wells 502 may be loaded using gravity or other loading technique (e.g., centrifugation, liquid handler, acoustic loading, optoelectronic, etc.). In some instances, at least one of the wells 502 contains a single sample molecule 506 (e.g., cell) and a single bead 504.

Reagents may be loaded into a well either sequentially or concurrently. In some cases, reagents are introduced to the device either before or after a particular operation. In some cases, reagents (which may be provided, in certain instances, in droplets, or beads) are introduced sequentially such that different reactions or operations occur at different steps. The reagents (or droplets, or beads) may also be loaded at operations interspersed with a reaction or operation step. For example, beads (or droplets) comprising reagents for fragmenting polynucleotides (e.g., restriction enzymes) and/or other enzymes (e.g., transposases, ligases, polymerases, etc.) may be loaded into the well or plurality of wells, followed by loading of droplets, or beads comprising reagents for attaching nucleic acid barcode molecules to a sample nucleic acid molecule. Reagents may be provided concurrently or sequentially with a sample, e.g., a cell or cellular components (e.g., organelles, proteins, nucleic acid molecules, carbohydrates, lipids, etc.). Accordingly, use of wells may be useful in performing multi-step operations or reactions.

As described elsewhere herein, the nucleic acid barcode molecules and other reagents may be contained within a bead, or droplet. These beads, or droplets may be loaded into a partition (e.g., a microwell) before, after, or concurrently with the loading of a cell, such that each cell is contacted with a different bead, or droplet. This technique may be used to attach a unique nucleic acid barcode molecule to nucleic acid molecules obtained from each cell. Alternatively or in addition to, the sample nucleic acid molecules may be attached to a support. For instance, the partition (e.g., microwell) may comprise a bead which has coupled thereto a plurality of nucleic acid barcode molecules. The sample nucleic acid molecules, or derivatives thereof, may couple or attach to the nucleic acid barcode molecules on the support. The resulting barcoded nucleic acid molecules may then be removed from the partition, and in some instances, pooled and sequenced. In such cases, the nucleic acid barcode sequences may be used to trace the origin of the sample nucleic acid molecule. For example, polynucleotides with identical barcodes may be determined to originate from the same cell or partition, while polynucleotides with different barcodes may be determined to originate from different cells or partitions.

The samples or reagents may be loaded in the wells or microwells using a variety of approaches. The samples (e.g., a cell, cell bead, or cellular component) or reagents (as described herein) may be loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson. The geometry, spacing between wells, density, and size of the microwells may be modified to accommodate a useful sample or reagent distribution; for instance, the size and spacing of the microwells may be adjusted such that the sample or reagents may be distributed in a super-Poissonian fashion.

In one particular non-limiting example, the microwell array or plate comprises pairs of microwells, in which each pair of microwells is configured to hold a droplet (e.g., comprising a single cell) and a single bead (such as those described herein, which may, in some instances, also be encapsulated in a droplet). The droplet and the bead (or droplet containing the bead) may be loaded simultaneously or sequentially, and the droplet and the bead may be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets comprising different reagents and/or samples, which are merged upon contact or upon application of a stimulus. In such instances, the droplet of one microwell of the pair can comprise reagents that may react with an agent in the droplet of the other microwell of the pair. For instance, one droplet can comprise reagents that are configured to release the nucleic acid barcode molecules of a bead contained in another droplet, located in the adjacent microwell. Upon merging of the droplets, the nucleic acid barcode molecules may be released from the bead into the partition (e.g., the microwell or microwell pair that are in contact), and further processing may be performed (e.g., barcoding, nucleic acid reactions, etc.). In cases where intact or live cells are loaded in the microwells, one of the droplets may comprise lysis reagents for lysing the cell upon droplet merging.

A droplet or bead may be partitioned into a well. The droplets may be selected or subjected to pre-processing prior to loading into a well. For instance, the droplets may comprise cells, and only certain droplets, such as those containing a single cell (or at least one cell), may be selected for use in loading of the wells. Such a pre-selection process may be useful in efficient loading of single cells, such as to obtain a non-Poissonian distribution, or to pre-filter cells for a selected characteristic prior to further partitioning in the wells. Additionally, the technique may be useful in obtaining or preventing cell doublet or multiplet formation prior to or during loading of the microwell.

In some instances, the wells can comprise nucleic acid barcode molecules attached thereto. The nucleic acid barcode molecules may be attached to a surface of the well (e.g., a wall of the well). The nucleic acid barcode molecules may be attached to a droplet or bead that has been partitioned into the well. The nucleic acid barcode molecule (e.g., a partition barcode sequence) of one well may differ from the nucleic acid barcode molecule of another well, which can permit identification of the contents contained with a single partition or well. In some cases, the nucleic acid barcode molecule can comprise a spatial barcode sequence that can identify a spatial coordinate of a well, such as within the well array or well plate. In some cases, the nucleic acid barcode molecule can comprise a unique molecular identifier for individual molecule identification. In some instances, the nucleic acid barcode molecules may be configured to attach to or capture a nucleic acid molecule within a sample or cell distributed in the well. For example, the nucleic acid barcode molecules may comprise a capture sequence that may be used to capture or hybridize to a nucleic acid molecule (e.g., RNA, DNA) within the sample. In some instances, the nucleic acid barcode molecules may be releasable from the microwell. In some instances, the nucleic acid barcode molecules may be releasable from the bead or droplet. For instance, the nucleic acid barcode molecules may comprise a chemical cross-linker which may be cleaved upon application of a stimulus (e.g., photo-, magnetic, chemical, biological, stimulus). The nucleic acid barcode molecules, which may be hybridized or configured to hybridize to a sample nucleic acid molecule, may be collected and pooled for further processing, which can include nucleic acid processing (e.g., amplification, extension, reverse transcription, etc.) and/or characterization (e.g., sequencing). In some instances nucleic acid barcode molecules attached to a bead or droplet in a well may be hybridized to sample nucleic acid molecules, and the bead with the sample nucleic acid molecules hybridized thereto may be collected and pooled for further processing, which can include nucleic acid processing (e.g., amplification, extension, reverse transcription, etc.) and/or characterization (e.g., sequencing). In such cases, the unique partition barcode sequences may be used to identify the cell or partition from which a nucleic acid molecule originated.

Characterization of samples within a well may be performed. Such characterization can include, in non-limiting examples, imaging of the sample (e.g., cell, cell bead, or cellular components) or derivatives thereof. Characterization techniques such as microscopy or imaging may be useful in measuring sample profiles in fixed spatial locations. For instance, when cells are partitioned, optionally with beads, imaging of each microwell and the contents contained therein may provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging may be used to characterize live cells in the wells, including, but not limited to: dynamic live-cell tracking, cell-cell interactions (when two or more cells are co-partitioned), cell proliferation, etc. Alternatively or in addition to, imaging may be used to characterize a quantity of amplification products in the well.

In operation, a well may be loaded with a sample and reagents, simultaneously or sequentially. When cells or cell beads are loaded, the well may be subjected to washing, e.g., to remove excess cells from the well, microwell array, or plate. Similarly, washing may be performed to remove excess beads or other reagents from the well, microwell array, or plate. In the instances where live cells are used, the cells may be lysed in the individual partitions to release the intracellular components or cellular analytes. Alternatively, the cells may be fixed or permeabilized in the individual partitions. The intracellular components or cellular analytes may couple to a support, e.g., on a surface of the microwell, on a solid support (e.g., bead), or they may be collected for further downstream processing. For instance, after cell lysis, the intracellular components or cellular analytes may be transferred to individual droplets or other partitions for barcoding. Alternatively, or in addition to, the intracellular components or cellular analytes (e.g., nucleic acid molecules) may couple to a bead comprising a nucleic acid barcode molecule; subsequently, the bead may be collected and further processed, e.g., subjected to nucleic acid reaction such as reverse transcription, amplification, or extension, and the nucleic acid molecules thereon may be further characterized, e.g., via sequencing. Alternatively, or in addition to, the intracellular components or cellular analytes may be barcoded in the well (e.g., using a bead comprising nucleic acid barcode molecules that are releasable or on a surface of the microwell comprising nucleic acid barcode molecules). The barcoded nucleic acid molecules or analytes may be further processed in the well, or the barcoded nucleic acid molecules or analytes may be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful step, the well (or microwell array or plate) may be sealed (e.g., using an oil, membrane, wax, etc.), which enables storage of the assay or selective introduction of additional reagents.

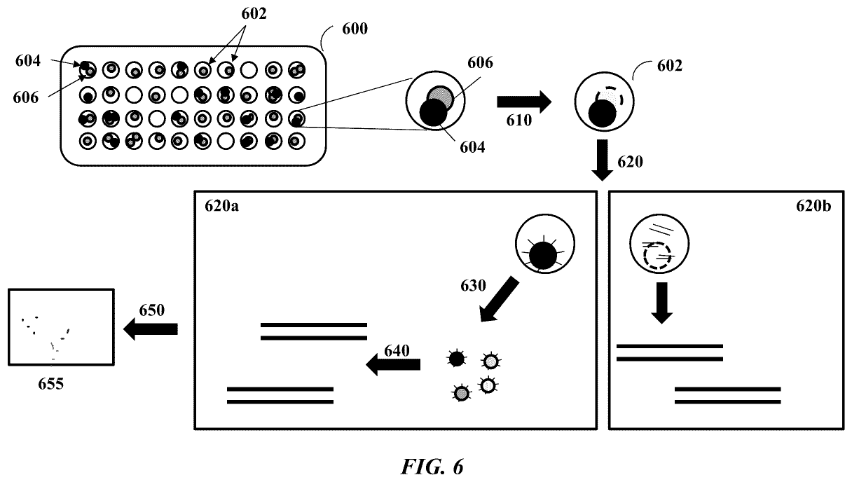

FIG. 6 schematically shows an example workflow for processing nucleic acid molecules within a sample. A substrate 600 comprising a plurality of microwells 602 may be provided. A sample 606 which may comprise a cell, cell bead, cellular components or analytes (e.g., proteins and/or nucleic acid molecules) can be co-partitioned, in a plurality of microwells 602, with a plurality of beads 604 comprising nucleic acid barcode molecules. During process 610, the sample 606 may be processed within the partition. For instance, in the case of live cells, the cell may be subjected to conditions sufficient to lyse the cells and release the analytes contained therein. In process 620, the bead 604 may be further processed. By way of example, processes 620a and 620b schematically illustrate different workflows, depending on the properties of the bead 604.

In 620a, the bead comprises nucleic acid barcode molecules that are attached thereto, and sample nucleic acid molecules (e.g., RNA, DNA) may attach, e.g., via hybridization of ligation, to the nucleic acid barcode molecules. Such attachment may occur on the bead. In process 630, the beads 604 from multiple wells 602 may be collected and pooled. Further processing may be performed in process 640. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 650, further characterization, such as sequencing may be performed to generate sequencing reads. The sequencing reads may yield information on individual cells or populations of cells, which may be represented visually or graphically, e.g., in a plot 655.

In 620b, the bead comprises nucleic acid barcode molecules that are releasably attached thereto, as described below. The bead may degrade or otherwise release the nucleic acid barcode molecules into the well 602; the nucleic acid barcode molecules may then be used to barcode nucleic acid molecules within the well 602. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 650, further characterization, such as sequencing may be performed to generate sequencing reads. The sequencing reads may yield information on individual cells or populations of cells, which may be represented visually or graphically, e.g., in a plot 655.

### Sample and Cell Processing

A sample may derive from any useful source including any subject, such as a human subject. A sample may comprise material (e.g., one or more biological particles) from one or more different sources, such as one or more different subjects. Multiple samples, such as multiple samples from a single subject (e.g., multiple samples obtained in the same or different manners from the same or different bodily locations, and/or obtained at the same or different times (e.g., seconds, minutes, hours, days, weeks, months, or years apparat)), or multiple samples from different subjects, may be obtained for analysis as described herein. For example, a first sample may be obtained from a subject at a first time and a second sample may be obtained from the subject at a second time later than the first time. The first time may be before a subject undergoes a treatment regimen or procedure (e.g., to address a disease or condition), and the second time may be during or after the subject undergoes the treatment regimen or procedure. In another example, a first sample may be obtained from a first bodily location or system of a subject (e.g., using a first collection technique) and a second sample may be obtained from a second bodily location or system of the subject (e.g., using a second collection technique), which second bodily location or system may be different than the first bodily location or system. In another example, multiple samples may be obtained from a subject at a same time from the same or different bodily locations. Different samples, such as different subjects collected from different bodily locations of a same subject, at different times, from multiple different subjects, and/or using different collection techniques, may undergo the same or different processing (e.g., as described herein). For example, a first sample may undergo a first processing protocol and a second sample may undergo a second processing protocol. In another example, a portion of a sample may undergo a first processing protocol and a second portion of the sample may undergo a second processing protocol.

A sample may be a biological sample, such as a cell sample (e.g., as described herein). A sample may include one or more biological particles, such as one or more cells and/or cellular constituents, such as one or more cell nuclei. A sample may be a tissue sample. For example, a sample may comprise a plurality of biological particles, such as a plurality of cells and/or cellular constituents. Biological particles (e.g., cells or cellular constituents, such as cell nuclei) of a sample may be of a single type or a plurality of different types. For example, cells of a sample may include one or more different types or blood cells.

Cells and cellular constituents of a sample may be of any type. For example, a cell or cellular constituent may be a vertebral, mammalian, fungal, plant, bacterial, or other cell type. In some cases, the cell is a mammalian cell, such as a human cell. The cell may be, for example, a stem cell, liver cell, nerve cell, bone cell, blood cell, reproductive cell, skin cell, skeletal muscle cell, cardiac muscle cell, smooth muscle cell, hair cell, hormone-secreting cell, or glandular cell. The cell may be, for example, an erythrocyte (e.g., red blood cell), a megakaryocyte (e.g., platelet precursor), a monocyte (e.g., white blood cell), a leukocyte, a B cell, a T cell (such as a helper, suppressor, cytotoxic, or natural killer T cell), an osteoclast, a dendritic cell, a connective tissue macrophage, an epidermal Langerhans cell, a microglial cell, a granulocyte, a hybridoma cell, a mast cell, a natural killer cell, a reticulocyte, a hematopoietic stem cell, a myoepithelial cell, a myeloid-derived suppressor cell, a platelet, a thymocyte, a satellite cell, an epithelial cell, an endothelial cell, an epididymal cell, a kidney cell, a liver cell, an adipocyte, a lipocyte, or a neuron cell. In some cases, the cell may be associated with a cancer, tumor, or neoplasm. In some cases, the cell may be associated with a fetus. In some cases, the cell may be a Jurkat cell.

A biological sample may include a plurality of cells having different dimensions and features. In some cases, processing of the biological sample, such as cell separation and sorting (e.g., as described herein), may affect the distribution of dimensions and cellular features included in the sample by depleting cells having certain features and dimensions and/or isolating cells having certain features and dimensions.

A sample may undergo one or more processes in preparation for analysis (e.g., as described herein), including, but not limited to, filtration, selective precipitation, purification, centrifugation, permeabilization, isolation, agitation, heating, and/or other processes. For example, a sample may be filtered to remove a contaminant or other materials. In an example, a filtration process may comprise the use of microfluidics (e.g., to separate biological particles of different sizes, types, charges, or other features).

In an example, a sample comprising one or more cells may be processed to separate the one or more cells from other materials in the sample (e.g., using centrifugation and/or another process). In some cases, cells and/or cellular constituents of a sample may be processed to separate and/or sort groups of cells and/or cellular constituents, such as to separate and/or sort cells and/or cellular constituents of different types. Examples of cell separation include, but are not limited to, separation of white blood cells or immune cells from other blood cells and components, separation of circulating tumor cells from blood, and separation of bacteria from bodily cells and/or environmental materials. A separation process may comprise a positive selection process (e.g., targeting of a cell type of interest for retention for subsequent downstream analysis, such as by use of a monoclonal antibody that targets a surface marker of the cell type of interest), a negative selection process (e.g., removal of one or more cell types and retention of one or more other cell types of interest), and/or a depletion process (e.g., removal of a single cell type from a sample, such as removal of red blood cells from peripheral blood mononuclear cells).

Separation of one or more different types of cells may comprise, for example, centrifugation, filtration, microfluidic-based sorting, flow cytometry, fluorescence-activated cell sorting (FACS), magnetic-activated cell sorting (MACS), buoyancy-activated cell sorting (BACS), or any other useful method.

For example, a flow cytometry method may be used to detect cells and/or cellular constituents based on a parameter such as a size, morphology, or protein expression. Flow cytometry-based cell sorting may comprise injecting a sample into a sheath fluid that conveys the cells and/or cellular constituents of the sample into a measurement region one at a time. In the measurement region, a light source such as a laser may interrogate the cells and/or cellular constituents and scattered light and/or fluorescence may be detected and converted into digital signals. A nozzle system (e.g., a vibrating nozzle system) may be used to generate droplets (e.g., aqueous droplets) comprising individual cells and/or cellular constituents. Droplets including cells and/or cellular constituents of interest (e.g., as determined via optical detection) may be labeled with an electric charge (e.g., using an electrical charging ring), which charge may be used to separate such droplets from droplets including other cells and/or cellular constituents. For example, FACS may comprise labeling cells and/or cellular constituents with fluorescent markers (e.g., using internal and/or external biomarkers). Cells and/or cellular constituents may then be measured and identified one by one and sorted based on the emitted fluorescence of the marker or absence thereof. MACS may use micro- or nano-scale magnetic particles to bind to cells and/or cellular constituents (e.g., via an antibody interaction with cell surface markers) to facilitate magnetic isolation of cells and/or cellular constituents of interest from other components of a sample (e.g., using a column-based analysis). BACS may use microbubbles (e.g., glass microbubbles) labeled with antibodies to target cells of interest. Cells and/or cellular components coupled to microbubbles may float to a surface of a solution, thereby separating target cells and/or cellular components from other components of a sample. Cell separation techniques may be used to enrich for populations of cells of interest (e.g., prior to partitioning, as described herein). For example, a sample comprising a plurality of cells including a plurality of cells of a given type may be subjected to a positive separation process. The plurality of cells of the given type may be labeled with a fluorescent marker (e.g., based on an expressed cell surface marker or another marker) and subjected to a FACS process to separate these cells from other cells of the plurality of cells. The selected cells may then be subjected to subsequent partition-based analysis (e.g., as described herein) or other downstream analysis. The fluorescent marker may be removed prior to such analysis or may be retained. The fluorescent marker may comprise an identifying feature, such as a nucleic acid barcode sequence and/or unique molecular identifier.

In another example, a first sample comprising a first plurality of cells including a first plurality of cells of a given type (e.g., immune cells expressing a particular marker or combination of markers) and a second sample comprising a second plurality of cells including a second plurality of cells of the given type may be subjected to a positive separation process. The first and second samples may be collected from the same or different subjects, at the same or different types, from the same or different bodily locations or systems, using the same or different collection techniques. For example, the first sample may be from a first subject and the second sample may be from a second subject different than the first subject. The first plurality of cells of the first sample may be provided a first plurality of fluorescent markers configured to label the first plurality of cells of the given type. The second plurality of cells of the second sample may be provided a second plurality of fluorescent markers configured to label the second plurality of cells of the given type. The first plurality of fluorescent markers may include a first identifying feature, such as a first barcode, while the second plurality of fluorescent markers may include a second identifying feature, such as a second barcode, that is different than the first identifying feature. The first plurality of fluorescent markers and the second plurality of fluorescent markers may fluoresce at the same intensities and over the same range of wavelengths upon excitation with a same excitation source (e.g., light source, such as a laser). The first and second samples may then be combined and subjected to a FACS process to separate cells of the given type from other cells based on the first plurality of fluorescent markers labeling the first plurality of cells of the given type and the second plurality of fluorescent markers labeling the second plurality of cells of the given type. Alternatively, the first and second samples may undergo separate FACS processes and the positively selected cells of the given type from the first sample and the positively selected cells of the given type from the second sample may then be combined for subsequent analysis. The encoded identifying features of the different fluorescent markers may be used to identify cells originating from the first sample and cells originating from the second sample. For example, the first and second identifying features may be configured to interact (e.g., in partitions, as described herein) with nucleic acid barcode molecules (e.g., as described herein) to generate barcoded nucleic acid products detectable using, e.g., nucleic acid sequencing.

### Multiplexing

The present disclosures provides methods and systems for multiplexing, and otherwise increasing throughput in, analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cell features may be used to characterize biological particles and/or cell features. In some instances, cell features include cell surface features. Cell surface features may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof. A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have a first reporter oligonucleotide coupled thereto, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, each of which is herein entirely incorporated by reference for all purposes.

In a particular example, a library of potential cell feature labelling agents may be provided, where the respective cell feature labelling agents are associated with nucleic acid reporter molecules, such that a different reporter oligonucleotide sequence is associated with each labelling agent capable of binding to a specific cell feature. In some aspects, different members of the library may be characterized by the presence of a different oligonucleotide sequence label. For example, an antibody capable of binding to a first protein may have associated with it a first reporter oligonucleotide sequence, while an antibody capable of binding to a second protein may have a different reporter oligonucleotide sequence associated with it. The presence of the particular oligonucleotide sequence may be indicative of the presence of a particular antibody or cell feature which may be recognized or bound by the particular antibody.

Labelling agents capable of binding to or otherwise coupling to one or more biological particles may be used to characterize a biological particle as belonging to a particular set of biological particles. For example, labeling agents may be used to label a sample of cells or a group of cells. In this way, a group of cells may be labeled as different from another group of cells. In an example, a first group of cells may originate from a first sample and a second group of cells may originate from a second sample. Labelling agents may allow the first group and second group to have a different labeling agent (or reporter oligonucleotide associated with the labeling agent). This may, for example, facilitate multiplexing, where cells of the first group and cells of the second group may be labeled separately and then pooled together for downstream analysis. The downstream detection of a label may indicate analytes as belonging to a particular group.

For example, a reporter oligonucleotide may be linked to an antibody or an epitope binding fragment thereof, and labeling a biological particle may comprise subjecting the antibody-linked barcode molecule or the epitope binding fragment-linked barcode molecule to conditions suitable for binding the antibody to a molecule present on a surface of the biological particle. The binding affinity between the antibody or the epitope binding fragment thereof and the molecule present on the surface may be within a desired range to ensure that the antibody or the epitope binding fragment thereof remains bound to the molecule. For example, the binding affinity may be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds may be less than about 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, or 1 pM. For example, the dissociation constant may be less than about 10 μM.

In another example, a reporter oligonucleotide may be coupled to a cell-penetrating peptide (CPP), and labeling cells may comprise delivering the CPP coupled reporter oligonucleotide into a biological particle. Labeling biological particles may comprise delivering the CPP conjugated oligonucleotide into a cell and/or cell bead by the cell-penetrating peptide. A cell-penetrating peptide that can be used in the methods provided herein can comprise at least one non-functional cysteine residue, which may be either free or derivatized to form a disulfide link with an oligonucleotide that has been modified for such linkage. Non-limiting examples of cell-penetrating peptides that can be used in embodiments herein include penetratin, transportan, plsl, TAT(48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The cell-penetrating peptide may be an arginine-rich peptide transporter. The cell-penetrating peptide may be Penetratin or the Tat peptide.

In another example, a reporter oligonucleotide may be coupled to a fluorophore or dye, and labeling cells may comprise subjecting the fluorophore-linked barcode molecule to conditions suitable for binding the fluorophore to the surface of the biological particle. In some instances, fluorophores can interact strongly with lipid bilayers and labeling biological particles may comprise subjecting the fluorophore-linked barcode molecule to conditions such that the fluorophore binds to or is inserted into a membrane of the biological particle. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLOS One. 2014 Feb. 4; 9 (2): e87649, which is hereby incorporated by reference in its entirety for all purposes, for a description of organic fluorophores.

A reporter oligonucleotide may be coupled to a lipophilic molecule, and labeling biological particles may comprise delivering the nucleic acid barcode molecule to a membrane of the biological particle or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and biological particle may be such that the biological particle retains the lipophilic molecule (e.g., and associated components, such as nucleic acid barcode molecules, thereof) during subsequent processing (e.g., partitioning, cell permeabilization, amplification, pooling, etc.). The reporter nucleotide may enter into the intracellular space and/or a cell nucleus.

A reporter oligonucleotide may be part of a nucleic acid molecule comprising any number of functional sequences, as described elsewhere herein, such as a target capture sequence, a random primer sequence, and the like, and coupled to another nucleic acid molecule that is, or is derived from, the analyte.

Prior to partitioning, the cells may be incubated with the library of labelling agents, that may be labelling agents to a broad panel of different cell features, e.g., receptors, proteins, etc., and which include their associated reporter oligonucleotides. Unbound labelling agents may be washed from the cells, and the cells may then be co-partitioned (e.g., into droplets or wells) along with partition-specific barcode oligonucleotides (e.g., attached to a support, such as a bead or gel bead) as described elsewhere herein. As a result, the partitions may include the cell or cells, as well as the bound labelling agents and their known, associated reporter oligonucleotides.

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent may interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. 20190323088, which is hereby entirely incorporated by reference for all purposes.

As described elsewhere herein, libraries of labelling agents may be associated with a particular cell feature as well as be used to identify analytes as originating from a particular biological particle, population, or sample. The biological particles may be incubated with a plurality of libraries and a given biological particle may comprise multiple labelling agents. For example, a cell may comprise coupled thereto a lipophilic labeling agent and an antibody. The lipophilic labeling agent may indicate that the cell is a member of a particular cell sample, whereas the antibody may indicate that the cell comprises a particular analyte. In this manner, the reporter oligonucleotides and labelling agents may allow multi-analyte, multiplexed analyses to be performed.

In some instances, these reporter oligonucleotides may comprise nucleic acid barcode sequences that permit identification of the labelling agent which the reporter oligonucleotide is coupled to. The use of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment), e.g., via a linker, using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2): 708-715, which is entirely incorporated herein by reference for all purposes. Likewise, protein and peptide biotinylation techniques have been developed and are readily available. Sec, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide may be allowed to hybridize to the reporter oligonucleotide.

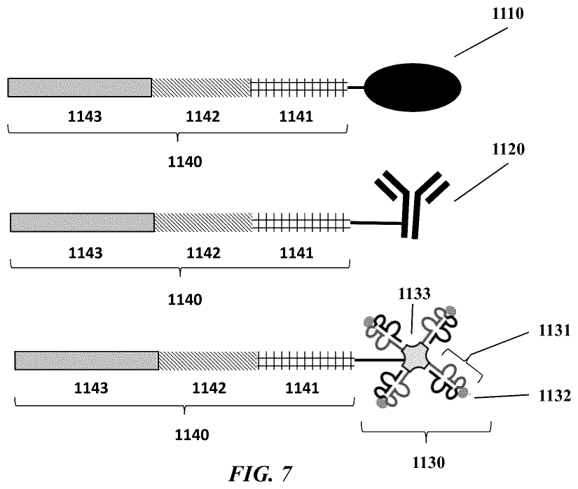

FIG. 7 describes exemplary labelling agents (1110, 1120, 1130) comprising reporter oligonucleotides (1140) attached thereto. Labelling agent 1110 (e.g., any of the labelling agents described herein) is attached (either directly, e.g., covalently attached, or indirectly) to reporter oligonucleotide 1140. Reporter oligonucleotide 1140 may comprise barcode sequence 1142 that identifies labelling agent 1110. Reporter oligonucleotide 1140 may also comprise one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

Referring to FIG. 7, in some instances, reporter oligonucleotide 1140 conjugated to a labelling agent (e.g., 1110, 1120, 1130) comprises a functional sequence 1141 (e.g., a primer sequence), a barcode sequence that identifies the labelling agent (e.g., 1110, 1120, 1130), and functional sequence 1143. Functional sequence 1143 can be a reporter capture handle sequence configured to hybridize to a complementary sequence, such as a complementary sequence present on a nucleic acid barcode molecule 1190 (not shown), such as those described elsewhere herein. In some instances, nucleic acid barcode molecule 1190 is attached to a support (e.g., a bead, such as a gel bead), such as those described elsewhere herein. For example, nucleic acid barcode molecule 1190 may be attached to the support via a releasable linkage (e.g., comprising a labile bond), such as those described elsewhere herein. In some instances, reporter oligonucleotide 1140 comprises one or more additional functional sequences, such as those described above.

In some instances, the labelling agent 1110 is a protein or polypeptide (e.g., an antigen or prospective antigen) comprising reporter oligonucleotide 1140. Reporter oligonucleotide 1140 comprises barcode sequence 1142 that identifies polypeptide 1110 and can be used to infer the presence of an analyte, e.g., a binding partner of polypeptide 1110 (i.e., a molecule or compound to which polypeptide 1110 can bind). In some instances, the labelling agent 1110 is a lipophilic moiety (e.g., cholesterol) comprising reporter oligonucleotide 1140, where the lipophilic moiety is selected such that labelling agent 1110 integrates into a membrane of a cell or nucleus. Reporter oligonucleotide 1140 comprises barcode sequence 1142 that identifies lipophilic moiety 1110 which in some instances is used to tag cells (e.g., groups of cells, cell samples, etc.) and may be used for multiplex analyses as described elsewhere herein. In some instances, the labelling agent is an antibody 1120 (or an epitope binding fragment thereof) comprising reporter oligonucleotide 1140. Reporter oligonucleotide 1140 comprises barcode sequence 1142 that identifies antibody 1120 and can be used to infer the presence of, e.g., a target of antibody 1120 (i.e., a molecule or compound to which antibody 1120 binds). In other embodiments, labelling agent 1130 comprises an MHC molecule 1131 comprising peptide 1132 and reporter oligonucleotide 1140 that identifies peptide 1132. In some instances, the MHC molecule is coupled to a support 1133. In some instances, support 1133 may be or comprise a polypeptide, such as streptavidin, avidin, neutravidin, or a polysaccharide, such as dextran. In some embodiments, support 1133 further comprises a detectable label, e.g., a detectable label described herein, e.g., a fluorescent label. In some instances, reporter oligonucleotide 1140 may be directly or indirectly coupled to MHC labelling agent 1130 in any suitable manner. For example, reporter oligonucleotide 1140 may be coupled to MHC molecule 1131, support 1133, or peptide 1132. In some embodiments, labelling agent 1130 comprises a plurality of MHC molecules described herein, (e.g. is an MHC multimer, which may be coupled to a support (e.g., 1133)). In some embodiments, reporter oligonucleotide 1140 and MHC molecule 1130 are attached to the polypeptide or polysaccharide of support 1133. In some embodiments, reporter oligonucleotide 1140 and MHC molecule 1130 are attached to the detectable label of support 1133. In some embodiments, reporter oligonucleotide 1140 and an antigen (e.g., protein, polypeptide) are attached to polypeptide or polysaccharide of support 1133. In some embodiments, reporter oligonucleotide 1140 and an antigen (e.g., protein, polypeptide) are attached to the detectable label of support 1133. There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labelling agents, including antibody and MHC-based labelling agents, reporter oligonucleotides, and methods of use, see, e.g., U.S. Pat. No. 10,550,429 and U.S. Pat. Pub. 20190367969, each of which is herein entirely incorporated by reference for all purposes.

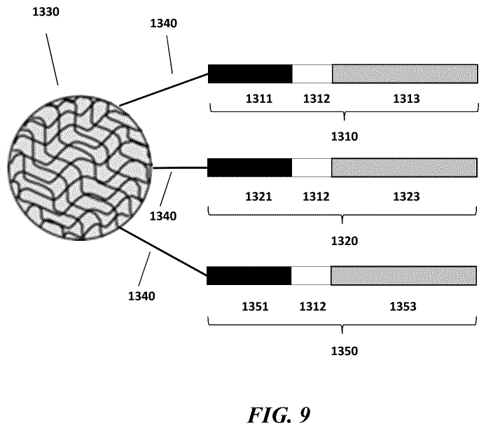

FIG. 9 illustrates another example of a barcode carrying bead. In some embodiments, analysis of multiple analytes (e.g., RNA and one or more analytes using labelling agents described herein) may comprise nucleic acid barcode molecules as generally depicted in FIG. 9. In some embodiments, nucleic acid barcode molecules 1310 and 1320 are attached to support 1330 via a releasable linkage 1340 (e.g., comprising a labile bond) as described elsewhere herein. Nucleic acid barcode molecule 1310 may comprise adapter sequence 1311, barcode sequence 1312 and capture sequence 1313. Nucleic acid barcode molecule 1320 may comprise adapter sequence 1321, barcode sequence 1312, and capture sequence 1323, wherein capture sequence 1323 comprises a different sequence than capture sequence 1313. In some instances, adapter 1311 and adapter 1321 comprise the same sequence. In some instances, adapter 1311 and adapter 1321 comprise different sequences. Although support 1330 is shown comprising nucleic acid barcode molecules 1310 and 1320, any suitable number of barcode molecules comprising common barcode sequence 1312 are contemplated herein. For example, in some embodiments, support 1330 further comprises nucleic acid barcode molecule 1350. Nucleic acid barcode molecule 1350 may comprise adapter sequence 1351, barcode sequence 1312 and capture sequence 1353, wherein capture sequence 1353 comprises a different sequence than capture sequence 1313 and 1323. In some instances, nucleic acid barcode molecules (e.g., 1310, 1320, 1350) comprise one or more additional functional sequences, such as a UMI or other sequences described herein. The nucleic acid barcode molecules 1310, 1320 or 1350 may interact with analytes as described elsewhere herein, for example, as depicted in FIGS. 12A-C.

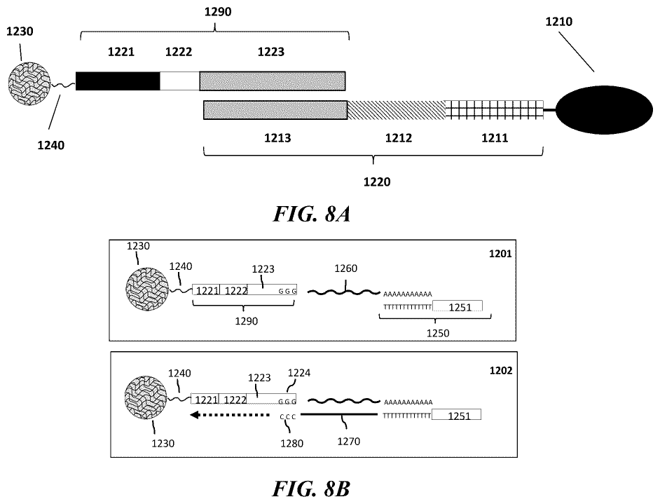

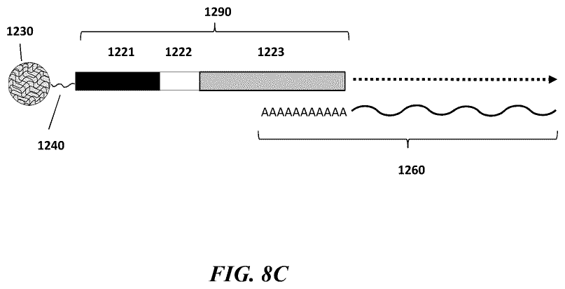

Referring to FIG. 8A, in an instance where cells are labelled with labeling agents, capture sequence 1223 may be complementary to an adapter sequence of a reporter oligonucleotide. Cells may be contacted with one or more reporter oligonucleotide 1220 conjugated labelling agents 1210 (e.g., polypeptide, antibody, or others described elsewhere herein). In some cases, the cells may be further processed prior to barcoding. For example, such processing steps may include one or more washing and/or cell sorting steps. In some instances, a cell that is bound to labelling agent 1210 which is conjugated to oligonucleotide 1220 and support 1230 (e.g., a bead, such as a gel bead) comprising nucleic acid barcode molecule 1290 is partitioned into a partition amongst a plurality of partitions (e.g., a droplet of a droplet emulsion or a well of a microwell array). In some instances, the partition comprises at most a single cell bound to labelling agent 1210. In some instances, reporter oligonucleotide 1220 conjugated to labelling agent 1210 (e.g., polypeptide, an antibody, pMHC molecule such as an MHC multimer, etc.) comprises a first adapter sequence 1211 (e.g., a primer sequence), a barcode sequence 1212 that identifies the labelling agent 1210 (e.g., the polypeptide, antibody, or peptide of a pMHC molecule or complex), and an capture handle sequence 1213. Capture handle sequence 1213 may be configured to hybridize to a complementary sequence, such as a capture sequence 1223 present on a nucleic acid barcode molecule 1290. In some instances, oligonucleotide 1220 comprises one or more additional functional sequences, such as those described elsewhere herein.

Barcoded nucleic may be generated (e.g., via a nucleic acid reaction, such as nucleic acid extension or ligation) from the constructs described in FIGS. 8A-C. For example, capture handle sequence 1213 may then be hybridized to complementary sequence, such as capture sequence 1223 to generate (e.g., via a nucleic acid reaction, such as nucleic acid extension or ligation) a barcoded nucleic acid molecule comprising cell (e.g., partition specific) barcode sequence 1222 (or a reverse complement thereof) and reporter barcode sequence 1212 (or a reverse complement thereof). In some embodiments, the nucleic acid barcode molecule 1290 (e.g., partition-specific barcode molecule) further includes a UMI (not shown). Barcoded nucleic acid molecules can then be optionally processed as described elsewhere herein, e.g., to amplify the molecules and/or append sequencing platform specific sequences to the fragments. Sec, e.g., U.S. Pat. Pub. 2018/0105808, which is hereby entirely incorporated by reference for all purposes. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) may be performed. For example, the workflow may comprise a workflow as generally depicted in any of FIGS. 8A-C, or a combination of workflows for an individual analyte, as described elsewhere herein. For example, by using a combination of the workflows as generally depicted in FIGS. 8A-C, multiple analytes can be analyzed.

In some instances, analysis of an analyte (e.g. a nucleic acid, a polypeptide, a carbohydrate, a lipid, etc.) comprises a workflow as generally depicted in FIG. 8A. A nucleic acid barcode molecule 1290 may be co-partitioned with the one or more analytes. In some instances, nucleic acid barcode molecule 1290 is attached to a support 1230 (e.g., a bead, such as a gel bead), such as those described elsewhere herein. For example, nucleic acid barcode molecule 1290 may be attached to support 1230 via a releasable linkage 1240 (e.g., comprising a labile bond), such as those described elsewhere herein. Nucleic acid barcode molecule 1290 may comprise a functional sequence 1221 and optionally comprise other additional sequences, for example, a barcode sequence 1222 (e.g., common barcode, partition-specific barcode, or other functional sequences described elsewhere herein), and/or a UMI sequence (not shown) . . . . The nucleic acid barcode molecule 1290 may comprise a capture sequence 1223 that may be complementary to another nucleic acid sequence, such that it may hybridize to a particular sequence, e.g., capture handle sequence 1213.

For example, capture sequence 1223 may comprise a poly-T sequence and may be used to hybridize to mRNA. Referring to FIG. 8C, in some embodiments, nucleic acid barcode molecule 1290 comprises capture sequence 1223 complementary to a sequence of RNA molecule 1260 from a cell. In some instances, capture sequence 1223 comprises a sequence specific for an RNA molecule. Capture sequence 1223 may comprise a known or targeted sequence or a random sequence. In some instances, a nucleic acid extension reaction may be performed, thereby generating a barcoded nucleic acid product comprising capture sequence 1223, the functional sequence 1221, barcode sequence 1222, any other functional sequence, and a sequence corresponding to the RNA molecule 1260.

In another example, capture sequence 1223 may be complementary to an overhang sequence or an adapter sequence that has been appended to an analyte. For example, referring to FIG. 8B, panel 1201, in some embodiments, primer 1250 comprises a sequence complementary to a sequence of nucleic acid molecule 1260 (such as an RNA encoding for a BCR sequence) from a biological particle. In some instances, primer 1250 comprises one or more sequences 1251 that are not complementary to RNA molecule 1260. Sequence 1251 may be a functional sequence as described elsewhere herein, for example, an adapter sequence, a sequencing primer sequence, or a sequence the facilitates coupling to a flow cell of a sequencer. In some instances, primer 1250 comprises a poly-T sequence. In some instances, primer 1250 comprises a sequence complementary to a target sequence in an RNA molecule. In some instances, primer 1250 comprises a sequence complementary to a region of an immune molecule, such as the constant region of a TCR or BCR sequence. Primer 1250 is hybridized to nucleic acid molecule 1260 and complementary molecule 1270 is generated (see Panel 1202). For example, complementary molecule 1270 may be cDNA generated in a reverse transcription reaction. In some instances, an additional sequence may be appended to complementary molecule 1270. For example, the reverse transcriptase enzyme may be selected such that several non-templated bases 1280 (e.g., a poly-C sequence) are appended to the cDNA. In another example, a terminal transferase may also be used to append the additional sequence. Nucleic acid barcode molecule 1290 comprises a sequence 1224 complementary to the non-templated bases, and the reverse transcriptase performs a template switching reaction onto nucleic acid barcode molecule 1290 to generate a barcoded nucleic acid molecule comprising cell (e.g., partition specific) barcode sequence 1222 (or a reverse complement thereof) and a sequence of complementary molecule 1270 (or a portion thereof). In some instances, sequence 1223 comprises a sequence complementary to a region of an immune molecule, such as the constant region of a TCR or BCR sequence. Sequence 1223 is hybridized to nucleic acid molecule 1260 and a complementary molecule 1270 is generated. For example complementary molecule 1270 may be generated in a reverse transcription reaction generating a barcoded nucleic acid molecule comprising cell (e.g., partition specific) barcode sequence 1222 (or a reverse complement thereof) and a sequence of complementary molecule 1270 (or a portion thereof). Additional methods and compositions suitable for barcoding cDNA generated from mRNA transcripts including those encoding V (D) J regions of an immune cell receptor and/or barcoding methods and composition including a template switch oligonucleotide are described in International Patent Application WO2018/075693, U.S. Patent Publication No. 2018/0105808, U.S. Patent Publication No. 2015/0376609, filed Jun. 26, 2015, and U.S. Patent Publication No. 2019/0367969, each of which applications is herein entirely incorporated by reference for all purposes.

In some embodiments, biological particles (e.g., cells, nuclei) from a plurality of samples (e.g., a plurality of subjects) can be pooled, sequenced, and demultiplexed by identifying mutational profiles associated with individual samples and mapping sequence data from single biological particles to their source based on their mutational profile. See, e.g., Xu J. et al., Genome Biology Vol. 20, 290 (2019); Huang Y. et al., Genome Biology Vol. 20, 273 (2019); and Heaton et al., Nature Methods volume 17, pages 615-620 (2020).

Gene expression data can reflect the underlying genome and mutations and structural variants therein. As a result, the variation inherent in the captured and sequenced RNA molecules can be used to identify genotypes de novo or used to assign molecules to genotypes that were known a priori. In some embodiments, allelic variation that is present due to haplotypic states (including linkage disequilibrium of the human leucocyte antigen loci (HLA), immune receptor loci (BCR), and other highly polymorphic regions of the genome), can also be used for demultiplexing. Expressed B cell receptors can be used to infer germline alleles from unrelated individuals, which information may be used for demultiplexing.

### Combinatorial Barcoding

In some instances, barcoding of a nucleic acid molecule may be done using a combinatorial approach. In such instances, one or more nucleic acid molecules (which may be comprised in a biological particle, e.g., a cell, e.g., a fixed cell, organelle, nucleus, or cell bead) may be partitioned (e.g., in a first set of partitions, e.g., wells or droplets) with one or more first nucleic acid barcode molecules (optionally coupled to a bead). The first nucleic acid barcode molecules or derivative thereof (e.g., complement, reverse complement) may then be attached to the one or more nucleic acid molecules, thereby generating barcoded nucleic acid molecules, e.g., using the processes described herein. The first nucleic acid barcode molecules may be partitioned to the first set of partitions such that a nucleic acid barcode molecule, of the first nucleic acid barcode molecules, that is in a partition comprises a barcode sequence that is unique to the partition among the first set of partitions. Each partition may comprise a unique barcode sequence. For example, a set of first nucleic acid barcode molecules partitioned to a first partition in the first set of partitions may each comprise a common barcode sequence that is unique to the first partition among the first set of partitions, and a second set of first nucleic acid barcode molecules partitioned to a second partition in the first set of partitions may each comprise another common barcode sequence that is unique to the second partition among the first set of partitions. Such barcode sequence (unique to the partition) may be useful in determining the cell or partition from which the one or more nucleic acid molecules (or derivatives thereof) originated.

The barcoded nucleic acid molecules from multiple partitions of the first set of partitions may be pooled and re-partitioned (e.g., in a second set of partitions, e.g., one or more wells or droplets) with one or more second nucleic acid barcode molecules. The second nucleic acid barcode molecules or derivative thereof may then be attached to the barcoded nucleic acid molecules. As with the first nucleic acid barcode molecules during the first round of partitioning, the second nucleic acid barcode molecules may be partitioned to the second set of partitions such that a nucleic acid barcode molecule, of the second nucleic acid barcode molecules, that is in a partition comprises a barcode sequence that is unique to the partition among the second set of partitions. Such barcode sequence may also be useful in determining the cell or partition from which the one or more nucleic acid molecules or first barcoded nucleic acid molecules originated. The barcoded nucleic acid molecules may thus comprise two barcode sequences (e.g., from the first nucleic acid barcode molecules and the second nucleic acid barcode molecules).

Additional barcode sequences may be attached to the barcoded nucleic acid molecules by repeating the processes any number of times (e.g., in a split-and-pool approach), thereby combinatorically synthesizing unique barcode sequences to barcode the one or more nucleic acid molecules. For example, combinatorial barcoding may comprise at least 1, 2, 3, 4, 5, 6, 7, 8, 9, 10 or more operations of splitting (e.g., partitioning) and/or pooling (e.g., from the partitions). Additional examples of combinatorial barcoding may also be found in International Patent Publication Nos. WO2019/165318, each of which is herein entirely incorporated by reference for all purposes.

Beneficially, the combinatorial barcode approach may be useful for generating greater barcode diversity, and synthesizing unique barcode sequences on nucleic acid molecules derived from a cell or partition. For example, combinatorial barcoding comprising three operations, each with 100 partitions, may yield up to 106 unique barcode combinations. In some instances, the combinatorial barcode approach may be helpful in determining whether a partition contained only one cell or more than one cell. For instance, the sequences of the first nucleic acid barcode molecule and the second nucleic acid barcode molecule may be used to determine whether a partition comprised more than one cell. For instance, if two nucleic acid molecules comprise different first barcode sequences but the same second barcode sequences, it may be inferred that the second set of partitions comprised two or more cells.

In some instances, combinatorial barcoding may be achieved in the same compartment. For instance, a unique nucleic acid molecule comprising one or more nucleic acid bases may be attached to a nucleic acid molecule (e.g., a sample or target nucleic acid molecule) in successive operations within a partition (e.g., droplet or well) to generate a barcoded nucleic acid molecule. A second unique nucleic acid molecule comprising one or more nucleic acid bases may be attached to the barcoded nucleic acid molecule. In some instances, all the reagents for barcoding and generating combinatorially barcoded molecules may be provided in a single reaction mixture, or the reagents may be provided sequentially.

In some instances, cell beads comprising nucleic acid molecules may be barcoded. Methods and systems for barcoding cell beads are further described in PCT/US2018/067356 and U.S. Pat. Pub. No. 2019/0330694, which are hereby incorporated by reference in its entirety.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

1. A method comprising:
a. partitioning a reaction mixture, or a portion thereof, into a plurality of partitions, wherein the reaction mixture comprises:
(i) a plurality of pre-determined antigen binding molecule (PABM)-associated beads, wherein a first PABM-associated bead of the plurality of PABM-associated beads comprises a first PABM, and a first PABM reporter oligonucleotide comprising a first PABM barcode sequence identifying the first PABM, and
(ii) a plurality of antigens comprising a first antigen operatively coupled to a first antigen reporter oligonucleotide comprising a first antigen reporter barcode sequence, wherein the first antigen binds to the first PABM of the PABM-associated bead,
and wherein the partitioning provides:
(i) a first partition comprising the first PABM-associated bead,
(ii) the first antigen, and
(iii) a plurality of first partition nucleic acid barcode molecules comprising a first partition-specific barcode sequence; and
b. generating
(i) a first barcoded polynucleotide comprising (i) the first partition barcode sequence or a reverse complement thereof and (ii) the first antigen reporter barcode sequence or reverse complement thereof; and
(ii) a second barcoded polynucleotide comprising (i) the first partition barcode sequence or reverse complement thereof and (ii) the first PABM reporter barcode sequence or reverse complement thereof.

2. The method of claim 1, wherein the reaction mixture further comprises a plurality of immune cells, wherein the plurality of immune cells comprises B cells and/or T cells.

3. The method of claim 1, wherein the partitioning further provides a second partition comprising (i) an immune cell expressing an antigen binding molecule (ABM), (ii) a target antigen operatively coupled to a target antigen reporter oligonucleotide comprising a target antigen barcode sequence, and (iii) a plurality of second partition nucleic acid barcode molecules comprising a second partition-specific barcode sequence.

4. The method of claim 3, wherein the ABM is selected from the group consisting of a T cell receptor (TCR), a B cell receptor (BCR), an antibody (Ab), and an antigen-binding fragment thereof.

5. The method of claim 3, wherein the target antigen:
(a) is of a pathogen, tumor, or an autoantigen, wherein the pathogen is a virus, bacteria or parasite,
(b) is different from the first antigen, and/or
(c) is the same as the first antigen.

6. The method of claim 3, wherein the first antigen:
(a) of a pathogen, tumor, or an autoantigen,
(b) is a growth factor or a growth factor receptor, and/or
(c) comprises a first MHC molecule bound to a first antigenic peptide.

7. The method of claim 3, wherein (b) further comprises generating (iii) a third barcoded polynucleotide comprising (i) the second partition barcode sequence or a reverse complement thereof and (ii) the target antigen reporter barcode sequence or a reverse complement thereof, and (iv) a fourth barcoded polynucleotide comprising (i) the second partition barcode sequence or a reverse complement thereof and (ii) a first nucleic acid sequence encoding at least a portion of the ABM expressed by the immune cell, or a reverse complement thereof.

8. The method of claim 3, wherein the plurality of PABM associated beads comprises a second PABM-associated bead, wherein the second PABM-associated bead comprises a second PABM and a second PABM reporter oligonucleotide comprising a second PABM barcode sequence identifying the second PABM, and wherein the partitioning provides (i) a 3^{rd} partition comprising the second PABM-associated bead, and (ii) a plurality of third partition nucleic acid barcode molecules comprising a third partition-specific barcode sequence, and wherein (b) further comprises generating a 5th barcoded polynucleotide comprising the 3^{rd} partition barcode sequence or reverse complement thereof and the 2^{nd} PABM reporter barcode sequence or reverse complement thereof.

9. The method of claim 8, wherein
(i) the second PABM binds the first antigen, the third partition further comprises the first antigen operatively coupled to the first antigen reporter oligonucleotide, and wherein (b) further comprises generating a 6th barcoded polynucleotide comprising the 3^{rd} partition barcode sequence or reverse complement thereof and the first antigen reporter barcode sequence or reverse complement thereof, or
(ii) the second PABM binds a second antigen, the third partition further comprises the second antigen operatively coupled to a second antigen reporter oligonucleotide comprising a second antigen reporter barcode sequence, and wherein (b) further comprises generating a 6th barcoded polynucleotide comprising the 3^{rd} partition barcode sequence or reverse complement thereof and the second antigen reporter barcode sequence or reverse complement thereof.

10. The method of claim 9, wherein (i) the first PABM binds to the first antigen with a known binding characteristic and/or (ii) the second PABM binds to the first antigen or the second antigen with a known binding characteristic.

11. The method of claim 10, further comprising generating a count matrix, wherein the count matrix includes (i) an association of partition barcode sequences with the first PABM barcode sequences and a quantity of first antigen barcode sequences and/or (ii) an association of partition barcode sequences with the second PABM barcode sequences and quantity of first or second antigen barcode sequences.

12. The method of claim 11, further comprising using the count matrix to correlate (i) the known binding characteristic of the first PABM for the first antigen with the quantity of first antigen barcode sequences and/or (ii) the known binding characteristic of the second PABM for the first antigen or the second antigen with the quantity of first or second antigen barcode sequences.

13. The method of claim 12, wherein the known binding characteristic of the second PABM for the first antigen or the second antigen is different from that of the first PABM for the first antigen.

14. The method of claim 12, wherein the correlations between the known binding characteristics of the 1^{st} and 2^{nd} PABMs with their associated quantities of 1^{st} or 2^{nd} antigen barcode sequences is used to infer a relationship between antigen barcode sequence quantity and the binding characteristic.

15. The method of claim 9, wherein the reporter oligonucleotide(s) and/or nucleic acid barcode molecule(s) comprise a unique molecule identifier (UMI).

16. The method of claim 15, wherein (i) the count matrix further includes an association of partition barcode sequences with target antigen UMI counts and nucleic acid sequences encoding the ABM expressed by the immune cell, or (ii) the method further comprises generating a count matrix that includes an association of partition barcode sequences with target antigen UMI counts and nucleic acid sequences encoding the ABM expressed by the immune cell.

17. The method of claim 14, wherein the method further comprises using the inferred relationship to estimate the binding characteristic of the ABM for the target antigen.

18. The method of claim 3, wherein a first nucleic acid barcode molecule of the plurality of second partition nucleic acid barcode molecules further comprises a capture sequence configured to couple to: (a) an mRNA or DNA analyte or (b) non-templated nucleotides appended to a cDNA reverse transcribed from an mRNA analyte.

19. The method of claim 7, further comprising determining the nucleic acid sequence encoding at least the portion of the ABM from the fourth barcoded polynucleotide.

20. The method of claim 10, wherein the known binding characteristic comprises a known binding affinity, a known on-rate, and/or a known off-rate.

21. A system for characterizing an ABM, the system comprising:
(a) a first pre-determined antigen binding molecule (PABM)-associated bead, wherein the first PABM-associated bead comprises a first PABM, and a first PABM reporter oligonucleotide.
(b) a second pre-determined antigen binding molecule (PABM)-associated bead, wherein the second PABM-associated bead comprises a second PABM, and a second PABM reporter oligonucleotide.
(c) a target antigen.

22. A kit comprising:
(a) a plurality of pre-determined antigen binding molecule (PABM)-associated beads, each comprising a PABM barcode sequence, and
(b) instructions for use.
